SEQUENCE LISTING (2 sequences)

SEQ ID NO: 1 - DNA, 3245 bp, Homo sapiens
  CDS (488)..(1795)
tatccacacacagcatcggaatattgctagctaactcaacaaatgtgcaaatcaggggactgttgtgtgtgtaccgattcatgtctagactgtttttattggtgaagtaggaactgcctcatcagtcatgggatcatagtgtcacagatggaaaagcaactatattagtctaaatatttgattctgcagttgcatgcaccaaattcagtgaggttagatgttaaatcatcttgttggctttgggctgaatttgatctaagagacaaaagtctcaaacaacagactacttactgccaccacatctcgattcaaagaatagttttcacatgttcgtggtgtggaaaggactttctgtttctcactaatttcttcagctataccaagagtggtgttgtctttgaacaggaaggacagcaaaaataaacataacagttttttcataagatacacagctgcattacacatcccaaaattgcttctctgcagaatgtcaaacaaagatcgacacattgattccagctgttcgtccttcatcaagacggaaccttccagcccagcctccctgacggacagcgtcaaccaccacagccctggtggctcttcagacgccagtgggagctacagttcaaccatgaatggccatcagaacggacttgactcgccacctctctacccttctgctcctatcctgggaggtagtgggcctgtcaggaaactgtatgatgactgctccagcaccattgttgaagatccccagaccaagtgtgaatacatgctcaactcgatgcccaagagactgtgtttagtgtgtggtgacatcgcttctgggtaccactatggggtagcatcatgtgaagcctgcaaggcattcttcaagaggacaattcaaggcaatatagaatacagctgccctgccacgaatgaatgtgaaatcacaaagcgcagacgtaaatcctgccaggcttgccgcttcatgaagtgtttaaaagtgggcatgctgaaagaaggggtgcgtcttgacagagtacgtggaggtcggcagaagtacaagcgcaggatagatgcggagaacagcccatacctgaaccctcagctggttcagccagccaaaaagccatataacaagattgtctcacatttgttggtggctgaaccggagaagatctatgccatgcctgaccctactgtccccgacagtgacatcaaagccctcactacactgtgtgacttggccgaccgagagttggtggttatcattggatgggcgaagcatattccaggcttctccacgctgtccctggcggaccagatgagccttctgcagagtgcttggatggaaattttgatccttggtgtcgtataccggtctctttcatttgaggatgaacttgtctatgcagacgattatataatggacgaagaccagtccaaattagcaggccttcttgatctaaataatgctatcctgcagctggtaaagaaatacaagagcatgaagctggaaaaagaagaatttgtcaccctcaaagctatagctcttgctaattcagactccatgcacatagaagatgttgaagccgttcagaagcttcaggatgtcttacatgaagcgctgcaggattatgaagctggccagcacatggaagaccctcgtcgagctggcaagatgctgatgacactgccactcctgaggcagacctctaccaaggccgtgcagcatttctacaacatcaaactagaaggcaaagtcccaatgcacaaactttttttggaaatgttggaggccaaggtctgactaaaagctccctgggccttcccatccttcatgttgaaaaagggaaaataaacccaagagtgatgtcgaagaaacttagagtttagttaacaacatcaaaaatcaacagactgcactgataatttagcagcaagactatgaagcagctttcagattcctccataggttcctgatgagttctttctactttctccatcatcttctttcctctttcttcccacatttctctttctctttattttttctccttttcttctttcacctcccttatttctttgcttctttcattcctagttcccattctcctttattttcttcccgtctgcctgccttctttcttttctttacctactctcattcctctcttttctcatccttccccttttttctaaatttgaaatagctttagtttaaaaaaaaaaatcctcccttccccctttcctttccctttctttcctttttccctttccttttccctttcctttcctttcctcttgaccttctttccatctttctttttcttccttctgctgctgaacttttaaaagaggtctctaactgaagagagatggaagccagccctgccaaaggatggagatccataatatggatgccagtgaacttattgtgaaccataccgtccccaatgactaaggaatcaaagagagagaaccaacgttcctaaaagtacagtgcaacatatacaaattgactgagtgcagtattagatttcatgggagcagcctctaattagacaacttaagcaacgttgcatcggctgcttcttatcattgcttttccatctagatcagttacagccatttgattccttaattgttttttcaagtcttccaggtatttgttagtttagctactatgtaactttttcagggaatagtttaagctttattcattcatgcaatactaaagagaaataagaatactgcaattttgtgctggctttgaacaattacgaacaataatgaaggacaaatgaatcctgaaggaagatttttaaaaatgttttgtttcttcttacaaatggagatttttttgtaccagctttaccacttttcagccatttattaatatgggaatttaacttactcaagcaatagttgaagggaaggtgcatattatcacggatgcaatttatgttgtgtgccagtctggtcccaaacatcaatttcttaacatgagctccagtttacctaaatgttcactgacacaaaggatgagattacacctacagtgactctgagtagtcacatatataagcactgcacatgagatatagatccgtagaattgtcaggagtgcacctctctacttgggaggtacaattgccatatgatttctagctgccatggtggttaggaatgtgatactgcctgtttgcaaagtcacagaccttgcctcagaaggagctgtgagccagtattcatttaag

SEQ ID NO: 2 - protein, 435 aa, Homo sapiens
MSNKDRHIDSSCSSFIKTEPSSPASLTDSVNHHSPGGSSDASGSYSSTMNGHQNGLDSPPLYPSAPILGGSGPVRKLYDDCSSTIVEDPQTKCEYMLNSMPKRLCLVCGDIASGYHYGVASCEACKAFFKRTIQGNIEYSCPATNECEITKRRRKSCQACRFMKCLKVGMLKEGVRLDRVRGGRQKYKRRIDAENSPYLNPQLVQPAKKPYNKIVSHLLVAEPEKIYAMPDPTVPDSDIKALTTLCDLADRELVVIIGWAKHIPGFSTLSLADQMSLLQSAWMEILILGVVYRSLSFEDELVYADDYIMDEDQSKLAGLLDLNNAILQLVKKYKSMKLEKEEFVTLKAIALANSDSMHIEDVEAVQKLQDVLHEALQDYEAGQHMEDPRRAGKMLMTLPLLRQTSTKAVQHFYNIKLEGKVPMHKLFLEMLEAKV

(19) United States
(12) Patent Application Publication
(10) Pub. No.: US 2018/0207114 A1
(43) Pub. Date: Jul. 26, 2018

(54) ESTROGEN RELATED RECEPTOR GAMMA (ERRgamma) ENHANCES AND MAINTAINS BROWN FAT THERMOGENIC CAPACITY

(71) Applicant: Salk Institute for Biological Studies, La Jolla, CA (US)

(72) Inventors: Ronald M. Evans, La Jolla, CA (US); Michael Downes, San Diego, CA (US); Annette Atkins, San Diego, CA (US); Ruth T. Yu, La Jolla, CA (US); Maryam Ahmadian, La Jolla, CA (US)

(73) Assignee: Salk Institute for Biological Studies, La Jolla, CA (US)

(21) Appl. No.: 15/927,232

(22) Filed: Mar. 21, 2018

Related U.S. Application Data
(63) Continuation of application No. PCT/US2016/050929, filed on Sep. 9, 2016.
(60) Provisional application No. 62/233,205, filed on Sep. 25, 2015.

(51) Int. Cl. A61K 31/166; A61K 31/381; A61K 31/40; A61K 31/519; A61K 31/44; A61K 31/137; A61K 31/7088; A61K 31/36; A61K 31/426; A61K 38/17; A61K 45/06; A61P 3/00

(52) U.S. Cl. CPC A61K 31/166; A61K 31/381; A61K 31/40; A61K 31/519; A61K 31/44; A61K 31/137; A61K 2300/00; A61K 31/36; A61K 31/426; A61K 38/177; A61K 45/06; A61P 3/00; A61K 31/7088

(57) ABSTRACT

Brown adipose tissue (BAT) plays a role in keeping an organism warm in response to a cold environment. In response to cold, transcription factors, including peroxisome proliferator receptor alpha (PGC1α), mediate the adaptive changes in the expression of oxidative and thermogenic genes in BAT. However, even without cold, BAT exhibits high expression of these genes relative to white adipose tissue (WAT). It is shown herein that estrogen related receptor gamma (ERRγ) is a critical factor that controls the expression of key metabolic genes in BAT under basal conditions. ERRγ is highly expressed in BAT versus WAT, yet is not transcriptionally induced by cold, suggesting it plays an important role in innate basal BAT function rather than in the adaptive response to cold. Based on these observations, methods of increasing thermogenesis in a subject by administering a therapeutically effective amount of one or more agents that increase ERRγ activity are provided.

### CROSS-REFERENCE TO RELATED APPLICATIONS

This application is a continuation of International Application No. PCT/US2016/050929, filed Sep. 9, 2016, which was published in English under PCT Article 21(2), which in turn claims priority to U.S. Provisional Application No. 62/233,205 filed Sep. 25, 2015, both herein incorporated by reference.

### ACKNOWLEDGMENT OF GOVERNMENT SUPPORT

This invention was made with government support under DK057978 awarded by The National Institutes of Health. The government has certain rights in the invention.

### FIELD

This application relates to methods of increasing thermogenesis in subjects in need thereof, by administering a therapeutically effective amount of one or more agents that increases ERRγ activity to the subject. Also provided are compositions that can be used for such methods.

### BACKGROUND

There are two types of adipose tissue, white adipose tissue (WAT) and brown adipose tissue (BAT). These two tissues differ from each other based on their gene expression signatures, morphology and physiological function (1). In contrast to WAT, BAT expresses high levels of genes involved in fatty acid oxidation and thermogenesis. BAT is also rich in mitochondria and has numerous small lipid droplets compared to the unilocular droplets of WAT (2, 3). As the major function of WAT is to store lipid, it is well equipped to adapt to fluctuations in nutrient availability. BAT, on the other hand, is specialized in burning lipid and is able to rapidly respond to changes in ambient temperature. While WAT has been extensively studied, much less is known about BAT since, until recently, it was not fully appreciated that adult humans have BAT (4-6).

BAT in humans is activated by cold and inversely correlated with obesity (4, 7). Recent evidence suggests that there are two types of thermogenic adipocytes in rodents with distinct developmental and anatomical features: classic brown adipocytes located in dedicated BAT depots, and beige adipocytes which reside mainly in subcutaneous WAT. Adult human BAT has characteristics of both rodent classic brown adipocytes and beige adipocytes, therefore understanding both of these cell types is critical (8-10). In contrast to beige adipocytes that arise postnatally in response to certain external cues, such as chronic cold exposure, brown adipocytes express relatively high amounts of thermogenic genes and remain primed for thermogenesis, even in the basal non-simulated state (11). While transcription factors and co-regulators, such as peroxisome proliferator-activated receptor gamma coactivator 1-alpha (PGC1α), mediate adaptive changes in the expression of oxidative and thermogenic genes in BAT in response to cold, factors that maintain this innate oxidative and thermogenic capacity in BAT, under the basal, thermoneutral state, are less understood (12, 13).

The estrogen-related receptor gamma (ERRγ) is an orphan nuclear receptor (NR) and member of the subfamily of Estrogen-Related Receptors, which also includes ERRα and β (15, 16). ERRγ is highly expressed in type 1 oxidative fibers compared to type 2 glycolytic skeletal muscle fibers. ERRγ maintains type 1 fibers in a highly oxidative state in the absence of exercise (14). ERRγ expression is high in BAT in comparison to WAT, but its in vivo role in BAT has never been investigated (14, 17-22).

### SUMMARY

It is shown herein that ERRγ is important for maintaining the basal oxidative and thermogenic capacity of BAT in the absence of cold stimulation. By binding to key thermogenic and oxidative genes, ERRγ is critical for maintaining basal BAT function. It is shown that loss of ERRγ in BAT leads to decreased expression of BAT signature genes under thermoneutral conditions resulting in a loss of a BAT phenotype and inability to survive when acutely exposed to cold.

Based on these observations methods of increasing thermogenesis in a subject, such as a human or veterinary subject having BAT, are provided. In one example, the methods include administering a therapeutically effective amount of one or more agents that increase ERRγ activity, thereby increasing thermogenesis in the subject. In some examples, the method decreases the body mass index (BMI) of an obese subject. In some examples, the method increases fatty acid uptake and/or oxidation in BAT. In some examples, the subject treated is one who is obese (e.g., has a BMI of at least 25, at least 30, such as 25-30, 30-35, 35-40 or over 40) or has reduced thermogenesis due to increased age. In some examples, the subject is at least 65 years old, at least 70 years old, or at least 75 years old. In some examples, the subject has or is at risk for hypothermia.

Examples of agents that can be used to increase ERRγ activity include a nucleic acid molecule encoding ERRγ (such as one having at least 80%, at least 90%, or at least 90% sequence identity to SEQ ID NO: 1), an ERRγ protein or active fragment thereof (such as one having at least 80%, at least 90%, or at least 90% sequence identity to SEQ ID NO: 2), ERRγ agonists (such as DY131, DY159, DY163, DY164, and GSK4716), or combinations thereof.

Also provided are compositions that include one or more agents that increase ERRγ activity (such as those provided herein) and one or more beta adrenergic agonists (such as those provided herein).

The foregoing and other objects and features of the disclosure will become more apparent from the following detailed description, which proceeds with reference to the accompanying figures.

### BRIEF DESCRIPTION OF THE DRAWINGS

FIGS. 1A-1H. ERRγ is highly expressed in mature brown adipocytes but is not induced by chronic cold. (A) Relative ERRγ mRNA levels in WAT and BAT of mice housed at thermoneutrality (30° C.) (n=10). (B) Relative ERRγ mRNA levels expression in differentiated human white adipose derived stem cells (ADSCs) and PAZ-6 human brown adipocytes (n=6). (C) Relative ERRγ and UCP-1 mRNA levels in the stromal vascular fraction (SVF) and mature adipocyte fraction of BAT from mice housed at thermoneutrality (30° C.) (n=3). (D) Relative ERRγ mRNA levels during the differentiation of PAZ-6 human brown adipocytes (n=4-6). (E) Relative mRNA levels of PGC1α, ERRα and ERRγ in BAT at thermoneutrality (TN) or cold-acclimated conditions (n=10). (F) Pearson correlation of the expression of Ppargc1a, Cox7a1 and UCP1 with ERRγ in BAT from 38 BXD strains of mice. (G) Relative ERRγ mRNA levels from BAT of control flox/flox (WT) and ERRγ ASKO (KO) mice (n=4-7). (H) Western blot for ERRγ and HDAC1 from nuclear extracts of WT and KO BAT.

FIGS. 2A-2I. (A) Relative ERRγ mRNA levels from the liver of control flox/flox (WT) and ERRγ ASKO (KO) mice (n=4-7). (B) Body weight of WT and KO mice on a chow diet housed at room temperature (22° C.) (n=8-13). (C) Body temperature of WT (left bar) and KO (right bar) mice on a chow diet housed at room temperature (22° C.) (n=7-10). (D) Lean mass of WT (left bar) and KO (right bar) mice on a chow diet housed at room temperature (22° C.) (n=7-10). (E) Fat mass of WT (left bar) and KO (right bar) mice on a chow diet housed at room temperature (22° C.) (n=7-10). (F) Fat pad weight of WT (left bar) and KO (right bar) mice on a chow diet housed at room temperature (22° C.) (n=7). (G) Organ weight of WT (left bar) and KO (right bar) mice on a chow diet housed at room temperature (22° C.) (n=7). (H) Glucose Tolerance Test (GTT) of WT and KO mice on a chow diet housed at room temperature (22° C.) (n=6-11). (I) Insulin Tolerance Test of WT and KO mice on a chow diet housed at room temperature (22° C.) (n=6-11).

FIGS. 3A-3E. ERRγ is required to maintain expression of thermogenic genes under basal conditions. (A) Upper: heatmap of selected BAT signature gene expression in BAT from ERRγ ASKO (KO) mice relative to BAT from flox/flox (WT) mice under cold acclimated (Cold), room temperature (RT) and thermoneutral (TN) conditions. Data are represented as log 2 fold change, n=3 per group. Lower: Schematic of temperature acclimation to cold and thermoneutral (TN) conditions. (B) Kyoto Encyclopedia of Genes and Genomes (KEGG) pathway analysis from all significantly down-regulated genes in KO BAT relative to WT BAT. (C) ERRγ occupancy on the Ucp1 and Fabp3 proximal promoters. (D) Heatmap showing the expression of BAT versus WAT selective genes at thermoneutrality in WT WAT, KO BAT and WT BAT. (E) Spearman correlation of Esrrg expression in the gene network of BAT- and WAT-selective genes in BAT of 38 strains of BXD mice.

FIGS. 4A-4G. (A) Representative RNA-Seq browser tracks of UCP-1 and leptin from WT and KO BAT from mice that were housed under TN or cold conditions. (B) Venn diagram showing unique and overlapping genes that are downregulated in ERRγ KO BAT and PPARa KO BAT from RNA-Seq. (C) Heatmap of selected BAT signature genes in BAT from PPARa KO mice (KO) mice relative to BAT from wild type (WT) and mice under thermoneutral conditions. Data are represented as log 2 fold change, n=3 per group. (D) Kyoto encyclopedia of Genes and Genomes (KEGG) pathway analysis from all significantly down-regulated genes in PPARa KO BAT relative to WT BAT. (E) Top enriched de novo binding motifs from ERRγ ChIP-Seq from BAT of WT mice acclimated to thermoneutrality. (F) Distribution of ERRγ occupancy from ERRγ ChIP-Seq from BAT of WT mice acclimated to thermoneutrality. (G) KEGG pathway analysis of all bound and downregulated genes from ChIP-Seq for ERRγ and RNA-Seq from ERRγ KO BAT, respectively.

FIGS. 5A-5M. (A) Body weights on a chow diet of male and female WT and KO mice housed at thermoneutrality. (B) Body weights on a high fat diet (HFD) of male and female WT and KO mice housed at thermoneutrality. (C) Fat mass and lean mass of 16 week old female mice housed at thermoneutrality on a chow and HFD. (D) Fat mass and lean mass of 16 week old male mice housed at thermoneutrality on a chow and HFD. (E) Serum levels of Leptin, Adiponectin, Triacylglycerol (TAG), Free Fatty Acids (FFA) and Insulin of male mice on a chow and HFD, housed at thermoneutrality. (F) Glucose Tolerance Test (GTT, left panel) and Insulin Tolerance Test (ITT, right panel) of male mice on a chow diet, housed at thermoneutrality. (G) Glucose Tolerance Test (GTT, left panel) and Insulin Tolerance Test (ITT, right panel) of male mice on a high fat diet, housed at thermoneutrality. (H) Organ weights of male WT and KO mice housed at thermoneutrality on a chow diet. (I) WAT depot weight of male WT and KO mice housed at thermoneutrality on a chow diet. (J) BAT weight of male WT and KO mice housed at thermoneutrality on a chow diet. (K) Mitochondrial size in BAT of male WT and KO mice housed at thermoneutrality on a chow diet. (L) Mitochondrial DNA to nuclear DNA ratio in BAT of male WT and KO mice housed at thermoneutrality on a chow diet. (M) Oxygen consumption rates measured in a Seahorse assay of differentiated PAZ-6 brown adipocytes treated overnight with vehicle (DMSO) or 10 μM of the ERRγ ligand GSK4716 in the presence or absence of palmitate.

FIGS. 6A-6H. ERRγ-ASKO mice exhibit a BAT to WAT phenotype with impaired fatty acid utilization. (A) Representative photograph of BAT from 10 week old control flox/flox (WT) and ERRγ ASKO (KO) mice housed at thermoneutrality since weaning. (B) Transmission electron microscopy of WT and KO BAT, focusing on the lipid droplets. (C) Quantitation of lipid droplet size in BAT. (D) Quantitation of TAG content in BAT. (E) FA acid uptake in differentiated PAZ-6 human brown adipocytes infected with ERRγ or control GFP adenovirus, with and without stimulation with a β adrenergic agonist. (F) FA acid oxidation in differentiated PAZ-6 human brown adipocytes infected with ERRγ or control GFP adenovirus, with and without stimulation with the β adrenergic agonist norepinephrine. (G) Oxygen consumption rate measured in seahorse assay from differentiated PAZ-6 brown adipocytes treated overnight with DMSO vehicle or 10 μM of GSK4716. (H) Quantitation of proton leak from (G).

FIGS. 7A-7F. ERRγ is required for survival during acute cold exposure. (A) Oxygen consumption (VO₂) after intraperitoneal injection of norepinephrine bitartrate (1 mg/kg) into control flox/flox (WT) and ERRγ ASKO (KO) mice housed at thermoneutrality (n=4-7). (B) VO₂ of WT and KO mice that were housed at thermoneutrality (TN) and then transferred to cold (4° C.) (n=6). (C) Body temperature of WT and KO mice exposed to cold (n=8-9). (D) Representative infrared image of WT and KO mice after exposure to cold. (E) Kaplan Meier survival curve of WT and KO mice during cold exposure (n=7-9). (F) Model depicting the role of ERRγ in maintaining basal BAT thermogenic capacity.

FIGS. 8A-8C. (A) VO₂ after intraperitoneal injection of CL316243 (1 mg/kg BW) from control flox/flox (WT) and ERRγ ASKO (KO) female mice housed at thermoneutrality (n=4). (B) Respiratory Exchange Ratio (RER) after intraperitoneal injection of CL316243 (1 mg/kg BW) from control flox/flox (WT) and ERRγ ASKO (KO) female mice housed at thermoneutrality (n=4). (C) BAT surface temperature measure with an infrared camera in WT and KO mice housed at thermoneutrality and then acutely exposed to the cold (4° C.).

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The sequence listing filed herewith is incorporated by reference (generated on Mar. 20, 2017 16 kb). In the accompanying sequence listing:

SEQ ID NOS: 1 and 2 are the nucleic acid and corresponding amino acid sequence of an exemplary human ERRγ sequence.

### DETAILED DESCRIPTION

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. “Comprising” means “including.” Hence “comprising A or B” means “including A” or “including B” or “including A and B.”

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references, including, for example, Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

All publications, patent applications, patents, and other references mentioned herein are incorporated by reference in their entirety for all purposes. All sequences associated with the GenBank® Accession numbers mentioned herein are incorporated by reference in their entirety as were present on Mar. 1, 2012. Although exemplary GenBank® numbers are listed herein, the disclosure is not limited to the use of these sequences. Many other ERRγ sequences are publicly available, and can thus be readily used in the disclosed methods. In one example, an ERRγ sequence has at least 70%, at least 75%, at least 80%, at least 85%, at least 90%, at least 95%, at least 98%, or at least 100% sequence identity to any of the GenBank® numbers are listed herein.

In order to facilitate review of the various embodiments of the disclosure, the following explanations of specific terms are provided:

Administration:

The introduction of a composition, such as an ERRγ agonist, into a subject by a chosen route, for example topically, orally, intravascularly such as intravenously, intramuscularly, intraperitoneally, intranasally, intradermally, transdermally, intrathecally, subcutaneously, via inhalation or via suppository. Administration can be local or systemic, such as intravenous or intramuscular. For example, if the chosen route is intravenous, the composition is administered by introducing the composition into a vein of the subject. In some examples an ERRγ agonist is administered to a subject at an effective dose.

Estrogen Receptor-Related Receptor γ (ERRγ):

(e.g., OMIM 602969) A constitutively active orphan nuclear receptor of the ERR subfamily. Unlike ERRα and β, it is more selectively expressed in metabolically active and highly vascularized tissues such as heart, kidney, and brain.

ERRγ sequences are publicly available. For example, GenBank® Accession Nos. NM_001134285.1, AY388461, AF058291.1 and NM_011935.2 disclose ERRγ nucleic acids, and GenBank® Accession Nos. NP_001127757.1, P62508.1, AAQ93381.1, and NP_{—β}36065.1 disclose ERRγ proteins. In certain examples, ERRγ has at least 80% sequence identity, for example at least 85%, at least 90%, at least 95%, at least 97%, at least 98%, or at least 99% sequence identity to such sequences (such as SEQ ID NO: 1 or 2), and retains ERRγ activity.

ERRγ activity includes the ability to promote thermogenesis, increase fatty acid update in brown adipose tissue, increase oxidation in brown adipose tissue, or combinations thereof.

Isolated:

An “isolated” biological component (such as a nucleic acid, protein or antibody) has been substantially separated, produced apart from, or purified away from other biological components in the cell of the organism in which the component naturally occurs, such as, other chromosomal and extrachromosomal DNA and RNA, and proteins. Nucleic acids and proteins which have been “isolated” thus include nucleic acids and proteins purified by standard purification methods. The term also embraces nucleic acids and proteins prepared by recombinant expression in a host cell as well as those chemically synthesized.

Pharmaceutically Acceptable Carriers:

The pharmaceutically acceptable carriers useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of an ERRγ agonist or other agent that increases ERRγ activity.

In general, the nature of the carrier will depend on the particular mode of administration being employed. For instance, parenteral formulations usually include injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Recombinant:

A recombinant nucleic acid molecule or protein is one that has a sequence that is not naturally occurring or has a sequence that is made by an artificial combination of two otherwise separated segments of sequence. This artificial combination can be accomplished by methods known in the art, such as chemical synthesis or by the artificial manipulation of isolated segments of nucleic acids, e.g., by genetic engineering techniques. Cells that express such molecules are referred to as recombinant or transgenic cells.

Sequence Identity:

The similarity between amino acid or nucleic acid sequences are expressed in terms of the similarity between the sequences, otherwise referred to as sequence identity. Sequence identity is frequently measured in terms of percentage identity (or similarity or homology); the higher the percentage, the more similar the two sequences are.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Variants of ERRγ that retain ERRγ activity are encompassed by this disclosure typically characterized by possession of at least about 75%, for example at least about 80%, at least 85%, at least 90%, at least 91%, at least 92%, at least 93%, at least 94%, at least 95%, at least 96%, at least 97%, at least 98% or at least 99% sequence identity counted over the full length alignment with the amino acid or nucleic acid sequence of interest, such as any of SEQ ID NOS: 1-2. Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Subject:

Living multi-cellular vertebrate organisms, a category that includes human and non-human mammals.

Therapeutically Effective Amount:

An amount of a pharmaceutical preparation that alone, or together with a pharmaceutically acceptable carrier or one or more additional therapeutic agents, induces the desired response. A therapeutic agent, such as an ERRγ agonist, is administered in therapeutically effective amounts. In some embodiments, a therapeutically effective amount is the amount of one or more agents that increase ERRγ activity necessary to increase one or more of thermogenesis, fatty acid uptake in brown adipose tissue (BAT), and/or oxidation in BAT (such as an increase of at least 20%, at least 50%, at least 60%, at least 75%, at least 80%, at least 90%, at least 95%, at least 100%, at least 200%, at least 300%, at least 400%, or at least 500%, as compared to an absence of the one or more agents that increase ERRγ activity). When administered to a subject, a dosage will generally be used that will achieve target tissue concentrations that has been shown to achieve a desired in vitro effect.

Effective amounts a therapeutic agent can be determined in many different ways, such as assaying for an increase in thermogenesis, fatty acid uptake and/or oxidation or improvement of physiological condition of a subject having or at risk for a disease such as obesity, hypothermia, and the like. Effective amounts also can be determined through various in vitro, in vivo or in situ assays.

Therapeutic agents can be administered in a single dose, or in several doses, for example daily, during a course of treatment. However, the effective amount of can be dependent on the source applied, the subject being treated, the severity and type of the condition being treated, and the manner of administration.

Treating a Disease:

“Treatment” refers to a therapeutic intervention that ameliorates a sign or symptom of a disease or pathological condition after it has begun to develop, such a sign or symptom of obesity, hypothermia, and the like. Treatment can also induce remission or cure of a condition. Preventing a disease refers to a therapeutic intervention to a subject who does not exhibit signs of a disease or exhibits only early signs for the purpose of decreasing the risk of developing pathology, such that the therapy inhibits or delays the full development of a disease, such as preventing development of obesity, hypothermia, and the like. Treatment and prevention of a disease does not require a total absence of disease. For example, a decrease of at least 20% or at least 50% can be sufficient. The beneficial effect can be evidenced, for example, by a delayed onset of clinical symptoms of the disease in a susceptible subject, a reduction in severity of some or all clinical symptoms of the disease, a slower progression of the disease, a reduction in the obesity or hypothermia, an improvement in the overall health or well-being of the subject, or by other parameters well known in the art that are specific to the particular disease.

Upregulated or Activation:

When used in reference to the expression of a nucleic acid molecule, such as an ERRγ gene, refers to any process which results in an increase in production of a gene product. A gene product can be RNA (such as mRNA, rRNA, tRNA, and structural RNA) or protein (such as an ERRγ protein). Therefore, gene upregulation or activation includes processes that increase transcription of a gene or translation of mRNA.

Examples of processes that increase transcription include those that facilitate formation of a transcription initiation complex, those that increase transcription initiation rate, those that increase transcription elongation rate, those that increase processivity of transcription and those that relieve transcriptional repression (for example by blocking the binding of a transcriptional repressor). Gene upregulation can include inhibition of repression as well as stimulation of expression above an existing level. Examples of processes that increase translation include those that increase translational initiation, those that increase translational elongation and those that increase mRNA stability.

Gene upregulation includes any detectable increase in the production of a gene product. In certain examples, production of a gene product increases by at least 2-fold, for example at least 3-fold or at least 4-fold, as compared to a control (such an amount of gene expression in an untreated cell, such as a cell not contacted with an agent that increases ERRγ activity).

Vector:

A nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements known in the art.

### Overview

Brown adipose tissue (BAT) plays a critical role in keeping an organism warm in response to a cold environment. In response to cold, transcription factors and regulators, including peroxisome proliferator-activated receptor gamma coactivator 1-alpha (PGC1α), mediate the adaptive changes in the expression of oxidative and thermogenic genes in BAT. However, even in the absence of cold, BAT exhibits high expression of these genes relative to white adipose tissue (WAT). Yet, what maintains this innate oxidative and thermogenic capacity in BAT is not well understood. Understanding how these fundamental differences between BAT and WAT are maintained can be used to develop therapeutic strategies to activate BAT.

Here, it is shown that estrogen-related receptor gamma (ERRγ) as a critical factor that controls the expression of key metabolic genes in BAT under basal conditions. ERRγ is highly expressed in BAT versus WAT, yet is not transcriptionally induced by cold, demonstrating it plays an important role in innate basal BAT function rather than in the adaptive response to cold. Loss of ERRγ in other organs such as the heart and brain results in decreased expression of genes involved in mitochondrial oxidative metabolism (17). It was observed that ERRγ binds and controls the expression of key thermogenic and oxidative genes in BAT under the basal, thermoneutral state. Mice lacking ERRγ specifically in adipose tissue (ERRyASKO mice) revealed minimal changes in thermogenic gene expression under chronic cold conditions. However, at thermoneutrality there was marked down-regulation of genes involved in fatty acid oxidation and thermogenesis. Thus, under thermoneutral conditions, ERRyASKO mice develop a whitening of BAT with thermogenic capacity. This defective BAT results in an inability to survive during acute cold exposure, revealing the role of ERRγ for priming BAT for thermogenesis.

Thus, in BAT, ERRγ predominantly controls genes involved in fatty acid utilization, thermogenesis and BAT identity. Loss of ERRγ in BAT results in downregulation of key BAT genes involved in fatty acid utilization and thermogenesis. As a result, BAT from ERRγ ASKO mice takes on a WAT-like appearance, at both the macroscopic and microscopic level. ChIP-Seq of ERRγ in BAT revealed that ERRγ binds to critical thermogenic and oxidative genes in BAT. In addition to binding to key metabolic genes, ERRγ also binds to genes that are highly enriched in BAT versus WAT, and ERRγ ASKO BAT exhibits reduced expression of BAT-selective genes. Taken together, these findings establish ERRγ as a critical factor in the maintenance of basal BAT function.

Physiologically, ERRγ ASKO mice exhibit an impaired thermogenic capacity and are unable to survive when acutely exposed to cold. Despite the drastic impairment in thermogenic capacity, ERRγ ASKO mice do not gain more weight on chow or high fat diet (HFD). This phenotype is reminiscent of adult mice lacking PRDM16 in BAT (Myf5-PRDM16 mice). In classic BAT, PRDM16 is a critical component of the transcriptional network that drives and maintains BAT identity (29, 30). However, despite a severely blunted thermogenic capacity, Myf5 PRDM16 knockout mice do not gain more weight than WT mice. Interestingly, brown adipocytes derived from Myf5 PRDM16 knockout BAT have decreased expression of ERRγ as well as BAT-selective ERRγ target genes, including UCP-1, Coxa1 and PPARα. It is possible that some of PRDM16 effects are mediated through ERRγ and also that these factors may synergize to maintain BAT identity.

Notably, changes in ERRγ ASKO BAT were observed under thermoneutral conditions and not under chronic cold-acclimated conditions. This indicates that ERRγ is not required for adaptive cold-induced thermogenesis. In this regard, ERRγ works independently of PGC1α in vitro in brown adipocytes to induce UCP-1 and fatty acid oxidation (31). Recently, Gadd45 gamma has been identified as a cold inducible activator of ERRγ in BAT (32). It is possible that ERRγ is found in basal state to prime BAT for thermogenesis and Gadd45 further activates ERRγ to enhances thermogenesis upon cold exposure. In this regard, key BAT transcription factors and co activators, like ERRα and PGC1α that are induced upon cold exposure, may compensate for loss of ERRγ in ERRγ ASKO BAT. However it is also possible that ERRγ is displaced by these factors upon adaptation to cold (12). Nevertheless, ERRγ is required for priming BAT for thermogenesis to handle acute bouts of cold and is therefore a critical factor that maintains BAT in its basal state by coordinately regulating genes involved in fatty acid utilization and thermogenesis. Consistent with its role in maintaining basal BAT thermogenic capacity, ERRγ is not induced by cold but is expressed at much higher levels in BAT than WAT. In skeletal muscle, ERRγ, which is highly expressed in type 1 oxidative fibers compared to type 2 glycolytic fibers, is required for maintaining type 1 fibers in a highly oxidative state in the absence of exercise (14). Therefore, ERRγ may have a broad role in controlling metabolism under innate rather than adaptive conditions.

With the realization that adult humans have BAT and that its quantity is inversely correlated with obesity, BAT has emerged as an important therapeutic target. However, most studies have focused on BAT under cold-stimulated conditions, and those examining basal BAT function have been performed at room temperature, which is below the thermoneutral zone for mice (33). The phenotype in ERRγ ASKO mice was observed only under thermoneutral conditions. Since humans are generally at thermoneutrality, studying BAT under this condition may have more relevance to BAT in humans (29). In humans and rodents, BAT function becomes impaired with obesity and increasing age. In rodents, this age-associated decline in thermogenesis has been associated with BAT atrophy and loss of UCP-1 activity (34). Therefore, modulation of ERRγ activity is a therapeutic strategy in these physiologic and pathologic conditions.

### Methods of Increasing Thermogenesis

Methods of increasing thermogenesis in a subject, such as a human or veterinary subject having BAT, are provided. In one example, the methods include administering a therapeutically effective amount of one or more agents that increase ERRγ activity, thereby increasing thermogenesis in the subject. In some examples, thermogenesis is increased by at least 20%, at least 30%, at least 40%, at least 50%, at least 60%, at least 70%, at least 80%, at least 90%, at least 100%, at least 200%, at least 300%, at least 400%, or at least 500%, for example as compared to an amount of thermogenesis present prior to administration of the one or more agents that increase ERRγ activity or an amount of thermogenesis without administration of the one or more agents that increase ERRγ activity.

In some examples, the method decreases the body mass index (BMI) of an obese subject. In some examples, BMI is reduced by at least 10%, at least 15%, at least 20%, at least 25%, at least 30%, at least 35%, at least 40%, at least 45%, or at least 50%, for example as compared to the subject's BMI prior to administration of the one or more agents that increase ERRγ activity or a subject's BMI without administration of the one or more agents that increase ERRγ activity.

In some examples, the method increases fatty acid uptake in BAT. In some examples, fatty acid uptake in BAT is increased by at least 20%, at least 30%, at least 40%, at least 50%, at least 60%, at least 70%, at least 80%, at least 90%, at least 100%, at least 200%, at least 300%, at least 400%, or at least 500%, for example as compared to an amount of fatty acid uptake in BAT present prior to administration of the one or more agents that increase ERRγ activity or an amount of fatty acid uptake in BAT without administration of the one or more agents that increase ERRγ activity.

In some examples, the method increases oxidation in BAT. In some examples, oxidation in BAT is increased by at least 20%, at least 30%, at least 40%, at least 50%, at least 60%, at least 70%, at least 80%, at least 90%, at least 100%, at least 200%, at least 300%, at least 400%, or at least 500%, for example as compared to an amount of oxidation in BAT present prior to administration of the one or more agents that increase ERRγ activity or an amount of oxidation in BAT without administration of the one or more agents that increase ERRγ activity.

In some examples, combinations of these effects are observed.

### Increasing ERRγ Activity

The present disclosure provides methods and pharmaceutical compositions for increasing thermogenesis, increasing fatty acid update in BAT, and/or increasing oxidation in BAT, by increasing ERRγ activity and thereby treating or preventing disorders associated with decreased thermogenesis, fatty acid update, or oxidation in BAT. ERRγ activity may be increased by increasing the amount of ERRγ protein being produced or by enhancing the activity of ERRγ protein. This can be achieved, for example, by administering a nucleotide sequence encoding for an ERRγ protein, an agent which enhances ERRγ expression, a substantially purified ERRγ protein, an ERRγ agonist, or combinations thereof. An ERRγ agonist includes compounds which increase the ERRγ activity in a cell or tissue.

Administration of ERRγ Proteins

In one example, ERRγ activity is increased by administering to the subject an ERRγ protein, such as a pharmaceutical composition containing such a protein. ERRγ protein sequences are known. For example, GenBank® Accession Nos. NP_001127757.1, P62508.1, AAQ93381.1, and NP_{—β}36065.1 disclose exemplary ERRγ protein sequences. However, one skilled in the art will appreciate that variations of such proteins can also retain ERRγ activity. For example such variants may include one or more deletions, substitutions, or additions (or combinations thereof), such as 1-50 of such changes (such as 1-40, 1-30, 1-20, or 1-10 of such changes). In certain examples, a ERRγ protein has at least 80%, at least 85%, at least 90%, at least 95%, at least 97%, at least 98% or at least 99% sequence identity to such sequences (such as SEQ ID NO: 2), and retains ERRγ activity. In some examples, changes are not made to the ERRγ ligand binding domain (LBD). In some examples, residues Asp328, Arg316 and/or Asp275 are not changed. In some examples, a functional fragment of an ERRγ protein is used, such as one containing at least 50, at least 100, at least 150, at least 200, or at least 300 consecutive amino acids from an ERRγ protein.

One of skill will realize that variants of ERRγ proteins can be used, such as a variant containing conservative amino acid substitutions. Such conservative variants will retain critical amino acid residues necessary for ERRγ activity, and can retain the charge characteristics of the residues (e.g., in order to preserve the low pI and low toxicity of the molecules). Amino acid substitutions (such as at most one, at most two, at most three, at most four, at most five, or at most 10 amino acid substitutions, such as 1 to 10 or 1 to 5 conservative substitutions) can be made in an ERRγ protein sequence. Conservative amino acid substitution tables providing functionally similar amino acids are well known to one of ordinary skill in the art. The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

An ERRγ protein can be derivatized or linked to another molecule (such as another peptide or protein). For example, the ERRγ protein can be functionally linked (by chemical coupling, genetic fusion, noncovalent association or otherwise) to one or more other molecular entities, such as an antibody, a detection agent, or a pharmaceutical agent.

Methods of making proteins are routine in the art, for example by recombinant molecular biology methods or by chemical peptide synthesis. In one example, an ERRγ protein is expressed in a cell from a vector encoding the protein. In some examples, the expression vector encoding ERRγ also encodes a selectable marker. In some examples, the sequence encoding ERRγ also encodes a purification tag sequence (such as a His-tag, β-globin-tag or glutathione S-transferase- (GST) tag) at the N- or C-terminus of ERRγ, to assist in purification of the protein.

For example, expression of nucleic acids encoding ERRγ proteins can be achieved by operably linking the ERRγ DNA or cDNA to a promoter (which is either constitutive or inducible), followed by incorporation into an expression cassette. The promoter can be any promoter, such as a cytomegalovirus promoter or a human T cell lymphotrophic virus promoter (HTLV)-1. Optionally, an enhancer, such as a cytomegalovirus enhancer, is included in the construct. The cassettes can be suitable for replication and integration in either prokaryotes (such as E. coli) or eukaryotes (such as yeast or a mammalian cell). Typical expression cassettes contain specific sequences useful for regulation of the expression of the DNA encoding the protein. For example, the expression cassettes can include appropriate promoters, enhancers, transcription and translation terminators, initiation sequences, a start codon (i.e., ATG) in front of a protein-encoding gene, splicing signal for introns, sequences for the maintenance of the correct reading frame of that gene to permit proper translation of mRNA, and stop codons. The vector can encode a selectable marker, such as a marker encoding drug resistance (for example, ampicillin or tetracycline resistance).

To obtain high level expression of ERRγ, expression cassettes can include a strong promoter to direct transcription, a ribosome binding site for translational initiation (internal ribosomal binding sequences), and a transcription/translation terminator. Exemplary control sequences include the T7, trp, lac, tac, trc, or lambda promoters, the control region of fd coat protein, a ribosome binding site, and can include a transcription termination signal. For eukaryotic cells, the control sequences can include a promoter and/or an enhancer derived from, for example, an immunoglobulin gene, HTLV, SV40, polyoma, adenovirus, retrovirus, baculovirus, simian virus, promoters derived from the promoter for 3-phosphoglycerate kinase, the promoters of yeast acid phosphatase, the promoter of the yeast alpha-mating factors or cytomegalovirus, and a polyadenylation sequence, and can further include splice donor and/or acceptor sequences (for example, CMV and/or HTLV splice acceptor and donor sequences). The cassettes can be transferred into the chosen host cell by well-known methods such as transformation or electroporation for E. coli and calcium phosphate treatment, electroporation or lipofection for mammalian cells. Cells transformed by the cassettes can be selected by resistance to antibiotics conferred by genes contained in the cassettes, such as the amp, gpt, neo and hyg genes.

When the host is a eukaryote, such methods of transfection of DNA as calcium phosphate coprecipitates, conventional mechanical procedures such as microinjection, electroporation, insertion of a plasmid encased in liposomes, or virus vectors may be used. Eukaryotic cells can also be cotransformed with polynucleotide sequences encoding ERRγ, and a second foreign DNA molecule encoding a selectable phenotype, such as the herpes simplex thymidine kinase gene. Another method is to use a eukaryotic viral vector, such as simian virus 40 (SV40), retrovirus, adenovirus, adeno-associated virus, Herpes virus, or bovine papilloma virus, to transiently infect or transform eukaryotic cells and express the protein (see for example, Eukaryotic Viral Vectors, Cold Spring Harbor Laboratory, Gluzman ed., 1982). One can readily use an expression system, such as plasmids and vectors, to produce proteins in cells including higher eukaryotic cells such as the COS, CHO, HeLa, fibroblast cell lines, lymphoblast cell lines, and myeloma cell lines.

Once expressed, the recombinant ERRγ protein can be purified according to standard procedures of the art, including ammonium sulfate precipitation, affinity columns, column chromatography, and the like (see, generally, R. Scopes, PROTEIN PURIFICATION, Springer-Verlag, N.Y., 1982). The recovered ERRγ protein need not be 100% pure. Once purified, partially or to homogeneity as desired, the ERRγ protein can be used therapeutically.

Modifications can be made to a nucleic acid encoding ERRγ without diminishing its biological activity. Some modifications can be made to facilitate the cloning, expression, or incorporation of ERRγ into a fusion protein. Such modifications are well known and include, for example, termination codons, a methionine added at the amino terminus to provide an initiation, site, additional amino acids placed on either terminus to create conveniently located restriction sites, or additional amino acids (such as poly His) to aid in purification steps.

In one example, ERRγ protein is synthesized by condensation of the amino and carboxyl termini of shorter fragments. Methods of forming peptide bonds by activation of a carboxyl terminal end (such as by the use of the coupling reagent N, N′-dicylohexylcarbodimide) are well known.

Expression of ERR′ in a Subject

In one example, ERRγ activity is increased by administering to the subject a nucleic acid molecule encoding an ERRγ protein. ERRγ coding sequences are known. For example, GenBank® Accession Nos. NM_001134285.1, AY388461, AF058291.1 and NM_011935.2 disclose exemplary ERRγ nucleic acid sequences. However, one skilled in the art will appreciate that variations of such sequences can also encode a protein with ERRγ activity. For example such variants may encode a protein with one or more deletions, substitutions, or additions (or combinations thereof), such as 1-50 of such changes (such as 1-40, 1-30, 1-20, or 1-10 of such changes). In certain examples, an ERRγ coding sequence has at least 80%, at least 85%, at least 90%, at least 95%, at least 97%, at least 98% or at least 99% sequence identity to such sequences (such as SEQ ID NO: 1), and encodes a protein having ERRγ activity. One of skill in the art can readily use the genetic code to construct a variety of functionally equivalent nucleic acids, such as nucleic acids which differ in sequence but which encode the same ERRγ protein sequence.

Nucleic acid sequences encoding an ERRγ protein can be prepared by any suitable method including, for example, cloning of appropriate sequences or by direct chemical synthesis by methods such as the phosphotriester method of Narang et al., Meth. Enzymol. 68:90-99, 1979; the phosphodiester method of Brown et al., Meth. Enzymol. 68:109-151, 1979; the diethylphosphoramidite method of Beaucage et al., Tetra. Lett. 22:1859-1862, 1981; the solid phase phosphoramidite triester method described by Beaucage & Caruthers, Tetra. Letts. 22(20):1859-1862, 1981, for example, using an automated synthesizer as described in, for example, Needham-VanDevanter et al., Nucl. Acids Res. 12:6159-6168, 1984; and, the solid support method of U.S. Pat. No. 4,458,066. Chemical synthesis produces a single stranded oligonucleotide. This can be converted into double stranded DNA by hybridization with a complementary sequence or by polymerization with a DNA polymerase using the single strand as a template. One of skill will recognize that longer sequences may be obtained by the ligation of shorter sequences.

ERRγ nucleic acids can be prepared by routine cloning techniques. Examples of appropriate cloning and sequencing techniques, and instructions sufficient to direct persons of skill through many cloning exercises are found in Sambrook et al., supra, Berger and Kimmel (eds.), supra, and Ausubel, supra. Product information from manufacturers of biological reagents and experimental equipment also provide useful information. Such manufacturers include the SIGMA Chemical Company (Saint Louis, Mo.), R&D Systems (Minneapolis, Minn.), Pharmacia Amersham (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemie AG, Buchs, Switzerland), Invitrogen (Carlsbad, Calif.), and Applied Biosystems (Foster City, Calif.), as well as many other commercial sources. Nucleic acids can also be prepared by amplification methods. Amplification methods include but are not limited to polymerase chain reaction (PCR), ligase chain reaction (LCR), transcription-based amplification system (TAS), and the self-sustained sequence replication system (3SR). A wide variety of cloning methods, host cells, and in vitro amplification methodologies are well known.

In some examples, it may only be necessary to introduce the ERRγ genetic or protein elements into certain cells or tissues. For example, introducing ERRγ into only muscle, such as skeletal or cardiac muscle (or even a particular muscle), or only into BAT, may be sufficient. However, in some instances, it may be more therapeutically effective and simple to treat all of the patient's cells, or more broadly disseminate the ERRγ nucleic acid or protein, for example by intravascular administration.

Nucleic acids encoding ERRγ can be introduced into the cells of a subject using routine methods, such as by using recombinant viruses (e.g., viral vectors) or by using naked DNA or DNA complexes (non-viral methods). Thus, in some embodiments, a method of increasing ERRγ activity is achieved by introducing a nucleic acid molecule coding for ERRγ into the subject. A general strategy for transferring genes into donor cells is disclosed in U.S. Pat. No. 5,529,774. The nucleic acid encoding ERRγ can be administered to the subject by any method which allows the recombinant nucleic acid to reach the appropriate cells. Exemplary methods include injection, infusion, deposition, implantation, and topical administration. Injections can be intradermal, intramuscular, iv, or subcutaneous.

In one example, an ERRγ coding sequence is introduced into a subject in a non-infectious form, such as naked DNA or liposome encapsulated DNA. Such molecules can be introduced by injection (such as intramuscular, iv, ip, pneumatic injection, or a gene gun), or other routine methods (such as oral or nasal). In one example, ERRγ coding sequence is part of a lipoplex, dendrimer, or inorganic nanoparticle to assist in its delivery.

In one example, viral vectors are used. Generally, such methods include cloning an ERRγ coding sequence into a viral expression vector, and that vector is then introduced into the subject to be treated. The virus infects the cells, and produces the ERRγ protein sequence in vivo, where it has its desired therapeutic effect. The nucleic acid sequence encoding ERRγ can be placed under the control of a suitable promoter. Suitable promoters which may be employed include, but are not limited to, the gene's native promoter; retroviral LTR promoter; adenoviral promoters, such as the adenoviral major late promoter; the cytomegalovirus (CMV) promoter; the Rous Sarcoma Virus (RSV) promoter; inducible promoters, such as the MMTV promoter; the metallothionein promoter; heat shock promoters; the albumin promoter; the histone promoter; the β-actin promoter; TK promoters; B19 parvovirus promoters; and the ApoAI promoter.

Exemplary viral vectors include, but are not limited to: pox viruses, recombinant vacciniavirus, retroviruses (such as lentivirus), replication-deficient adenovirus strains, adeno-associated virus, herpes simplex virus, or poliovirus.

Adenoviral vectors may include essentially the complete adenoviral genome. Alternatively, the adenoviral vector may be a modified adenoviral vector in which at least a portion of the adenoviral genome has been deleted. In one embodiment, the vector includes an adenoviral 5′ ITR; an adenoviral 3′ ITR; an adenoviral encapsidation signal; a DNA sequence encoding a therapeutic agent such as EDA1-II, dl or DL; and a promoter for expressing the DNA sequence encoding a therapeutic agent. The vector is free of at least the majority of adenoviral E1 and E3 DNA sequences, but is not necessarily free of all of the E2 and E4 DNA sequences, and DNA sequences encoding adenoviral proteins transcribed by the adenoviral major late promoter. Such a vector may be constructed according to standard techniques, using a shuttle plasmid which contains, beginning at the 5′ end, an adenoviral 5′ ITR, an adenoviral encapsidation signal, and an Ela enhancer sequence; a promoter (which may be an adenoviral promoter or a foreign promoter); a tripartite leader sequence, a multiple cloning site (which may be as herein described); a poly A signal; and a DNA segment which corresponds to a segment of the adenoviral genome. The DNA segment serves as a substrate for homologous recombination with a modified or mutated adenovirus, and may encompass, for example, a segment of the adenovirus 5′ genome no longer than from base 3329 to base 6246. The plasmid may also include a selectable marker and an origin of replication. The origin of replication may be a bacterial origin of replication. A desired DNA sequence encoding a therapeutic agent may be inserted into the multiple cloning site of the plasmid. The plasmid may be used to produce an adenoviral vector by homologous recombination with a modified or mutated adenovirus in which at least the majority of the E1 and E3 adenoviral DNA sequences have been deleted. Homologous recombination may be effected through co-transfection of the plasmid vector and the modified adenovirus into a helper cell line, such as 293 cells, by CaPO4 precipitation. The homologous recombination produces a recombinant adenoviral vector which includes DNA sequences derived from the shuttle plasmid between the Not I site and the homologous recombination fragment, and DNA derived from the E1 and E3 deleted adenovirus between the homologous recombination fragment and the 3′ ITR.

In one embodiment, the viral vector is a retroviral vector. Examples of retroviral vectors which may be employed include, but are not limited to, Moloney Murine Leukemia Virus, spleen necrosis virus, and vectors derived from retroviruses such as Rous Sarcoma Virus, Harvey Sarcoma Virus, avian leukosis virus, human immunodeficiency virus, lentivirus, myeloproliferative sarcoma virus, and mammary tumor virus. The vector can be a replication defective retrovirus particle. Retroviral vectors are useful as agents to effect retroviral-mediated gene transfer into eukaryotic cells. Retroviral vectors are generally constructed such that the majority of sequences coding for the structural genes of the virus are deleted and replaced by the gene(s) of interest. Most often, the structural genes (e.g., gag, pol, and env), are removed from the retroviral backbone using genetic engineering techniques known in the art. An ERRγ coding sequence can be incorporated into a proviral backbone using routine methods. In some examples, the structural genes of the retrovirus are replaced by an ERRγ gene which then is transcribed under the control of the viral regulatory sequences within the long terminal repeat (LTR). Retroviral vectors have also been constructed which can introduce more than one gene into target cells. Usually, in such vectors one gene is under the regulatory control of the viral LTR, while the second gene is expressed either off a spliced message or is under the regulation of its own, internal promoter. Alternatively, two genes may be expressed from a single promoter by the use of an Internal Ribosome Entry Site.

In one example, the viral vector is an adeno-associated virus (AAV). Gene therapy vectors using AAV can infect both dividing and non-dividing cells and persist in an extrachromosomal state without integrating into the genome of the host cell. In some examples, the rep and cap are removed from the DNA of the AAV. The ERRγ coding sequence together with a promoter to drive transcription is inserted between the inverted terminal repeats (ITR) that aid in concatamer formation in the nucleus after the single-stranded vector DNA is converted by host cell DNA polymerase complexes into double-stranded DNA.

The viral particles are administered in an amount effective to produce a therapeutic effect in a host. The exact dosage of viral particles to be administered is dependent upon a variety of factors, including the age, weight, and sex of the patient to be treated, and the nature and extent of the disease or disorder to be treated. The viral particles may be administered as part of a preparation having a titer of viral particles of at least 1×10⁵ pfu/ml, at least 1×10⁶ pfu/ml, at least 1×10⁷ pfu/ml, at least 1×10⁸ pfu/ml, at least 1×10⁹ pfu/ml, or at least 1×10¹⁰ pfu/ml, and in some examples not exceeding 2×10¹¹ pfu/ml. The viral particles can be administered in combination with a pharmaceutically acceptable carrier, for example in a volume up to 10 ml. The pharmaceutically acceptable carrier may be, for example, a liquid carrier such as a saline solution, protamine sulfate or Polybrene.

Agonists

An ERRγ agonist is an agent that induces or increases ERRγ activity or expression. Agonists of ERRγ are commercially available, and can be generated using routine methods. Specific examples are provided below, and in the Examples section. In some examples, the agonist is an agonist of ERRγ, but not ERRα or ERRβ. In some examples, the agonist is an agonist of ERRγ, as well as of ERRα and/or ERRβ.

ERRγ agonists are known in the art, and additional ERRγ agonists can be identified using known methods (e.g., see Zuercher et al., 2005, J. Med. Chem. 48(9):3107-9; Coward et al. 2001, Proc Natl Acad Sci USA. 8(15):8880-4; and Zhou et al., 1998, Mol. Endocrin. 12:1594-1604).

For example, phenolic acyl hydrazones GSK4716 (e.g., Santa Cruz Catalog # sc-203986) and GSK9089 (also known as DY131, see for example, U.S. Pat. No. 7,544,838) (N-[(E)-[4-(diethylamino)phenyl]methylideneamino]-4-hydroxybenzamide; e.g., Tocris Bioscience Catalog #2266 or Santa Cruz Catalog # sc203571) are agonists of ERRβ and ERRγ.

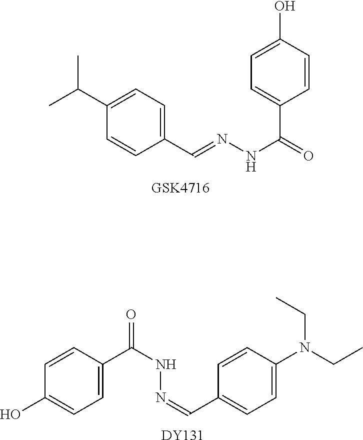 [SMILES: CC(C)c1ccc(/C=N/NC(=O)c2ccc(O)cc2)cc1 ; CCN(CC)c1ccc(/C=N\NC(=O)c2ccc(O)cc2)cc1 ; "DY131"]

Kim et al. (J. Comb. Chem. 11:928-37, 2009) disclose a screening assay for agonists of ERRγ derived from GSK4716. Such a screening method can also be used to identify other agonists of ERRγ. E6 was discovered as being selective for ERRγ but not ERRα and β.

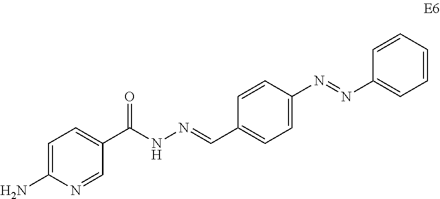 [SMILES: Nc1ccc(C(=O)N/N=C/c2ccc(/N=N/c3ccccc3)cc2)cn1]

U.S. Pat. Nos. 7,544,838 and 8,044,241 also provide ERRγ agonists that can be used with the disclosed methods, such as DY131. In addition, DY159, DY162, DY163 and DY164 were also observed to activate ERRγ (and ERRα and β), for example in the presence of PGC-1a.

US Patent Application Publication Nos. 2011/0218196 and 2009/0281191 also provide ERRγ agonists that can be used with the disclosed methods.

Subjects

Exemplary subjects that can benefit from the disclose therapies include human and veterinary mammalian subjects, such as cats, dogs, horses, rodents, and the like. In one example, the subject treated has, or is at risk for developing, decreased thermogenesis, decreased fatty acid update in BAT, and/or decreased oxidation in BAT. Thus, such therapies can be used to prevent or treat the disease provided herein. In some examples the patient is at risk for such diseases due to hypothermia, age, obesity, and the like, and thus such patients can be treated using the methods provided herein.

In some examples, the subject treated is one who is obese (e.g., has a BMI of at least 25, at least 30, such as 25-30, 30-35, 35-40 or over 40). In some examples, the subject treated is one who has reduced thermogenesis due to increased age. Thus, in some examples, the subject is at least 65 years old, at least 70 years old, or at least 75 years old. In some examples, the subject has or is at risk for hypothermia.

In some examples, a subject is monitored before, during and/or after treatment with one or more agents that increase ERRγ activity, such as measuring or determining the subject's body weight, body temperature, or both. Thus, in some examples the disclosed methods include such steps.

Administration of Agents that Increase ERRγ Activity

Compositions that include one or more agents that increase ERRγ activity, such as ERRγ nucleic acids, ERRγ proteins, and ERRγ agonists that can be used to increase thermogenesis, fatty acid uptake in BAT, and/or oxidation in BAT, are suited for the preparation of pharmaceutical compositions.

Pharmaceutical compositions that include one or more agents that increase ERRγ activity are provided. These pharmaceutical compositions can be used in methods of treatment/prevention of vascular, mitochondrial, and muscular disorders, and can be formulated with an appropriate physiologically acceptable solid or liquid carrier, depending upon the particular mode of administration chosen. A variety of aqueous carriers can be used, for example, buffered saline and the like. These solutions are sterile and generally free of undesirable matter. These compositions can be sterilized by conventional, well known sterilization techniques. The compositions can contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antibody in these formulations can vary widely, and will be selected primarily based on fluid volumes, viscosities, body weight and the like in accordance with the particular mode of administration selected and the subject's needs. Compositions including one or more agents that increase ERRγ activity are of use, for example, for the treatment of thermogenesis disorder, such as those resulting from obesity, age, and/or hypothermia.

The pharmaceutically acceptable carriers and excipients useful in this disclosure, for either therapeutic or diagnostic methods, are conventional. The one or more agents that increase ERRγ activity can be formulated for systemic or local (such as inhalational) administration. In one example, the one or more agents that increase ERRγ activity is formulated for parenteral administration, such as intravenous or intramuscular administration. For instance, parenteral formulations usually include injectable fluids that are pharmaceutically and physiologically acceptable fluid vehicles such as water, physiological saline, other balanced salt solutions, aqueous dextrose, glycerol or the like. Excipients that can be included are, for instance, other proteins, such as human serum albumin or plasma preparations. If desired, the pharmaceutical composition to be administered can also contain minor amounts of non-toxic auxiliary substances, such as wetting or emulsifying agents, preservatives, and pH buffering agents and the like, for example sodium acetate or sorbitan monolaurate.

The compositions can be prepared in unit dosage forms for administration to a subject. The dosage form of the pharmaceutical composition will be determined by the mode of administration chosen. For instance, in addition to injectable fluids, topical, inhalation, oral and suppository formulations can be employed. Topical preparations can include ointments, sprays and the like. Inhalation preparations can be liquid (such as solutions or suspensions) and include mists, sprays and the like. Oral formulations can be liquid (for example, syrups, solutions or suspensions), or solid (such as powders, pills, tablets, or capsules). Suppository preparations can also be solid, gel, or in a suspension form. For solid compositions, conventional non-toxic solid carriers can include pharmaceutical grades of mannitol, lactose, starch, or magnesium stearate. Actual methods of preparing such dosage forms are known, or will be apparent, to those skilled in the art.

The pharmaceutical compositions that include one or more agents that increase ERRγ activity can be formulated in unit dosage form suitable for individual administration of precise dosages. In addition, the pharmaceutical compositions may be administered in a single dose or as in a multiple dose schedule. A multiple dose schedule is one in which a primary course of treatment may be with more than one separate dose, for instance 1-10 doses, followed by other doses given at subsequent time intervals as needed to maintain or reinforce the action of the compositions. Treatment can involve daily or multi-daily doses of compound(s) over a period of a few days to months, or even years. Thus, the dosage regime will also, at least in part, be determined based on the particular needs of the subject to be treated, the severity of the affliction, whether the therapeutic agent is administered for preventive or therapeutic purposes, previous prophylaxis and therapy, the subject's clinical history and response to the therapeutic agent, and the manner of administration, and can be left to the judgment of the prescribing clinician. Within these bounds, the formulation to be administered will contain a quantity of the active component(s) in amounts effective to achieve the desired effect in the subject being treated. A therapeutically effective amount of one or more agents that increase ERRγ activity is one that which provides either subjective relief of a symptom(s) or an objectively identifiable improvement as noted by the clinician or other qualified observer. These compositions can be administered in conjunction with another agent, either simultaneously or sequentially. The one or more agents that increase ERRγ activity also can be used or administered as a mixture, for example in equal amounts, or individually, provided in sequence, or administered all at once.

Single or multiple administrations of the compositions can be administered depending on the dosage and frequency as required and tolerated by the subject. The composition should provide a sufficient quantity of one or more agents that increase ERRγ activity to effectively treat the subject or inhibit the development of the desired disease. The dosage can be administered once but can be applied periodically until either a therapeutic result is achieved or until side effects warrant discontinuation of therapy. In one example, a dose of the one or more agents that increase ERRγ activity is infused for thirty minutes every other day. In this example, about one to about ten doses can be administered, such as three or six doses can be administered every other day. In a further example, a continuous infusion is administered for about five to about ten days. The subject can be treated at regular intervals, such as monthly, until a desired therapeutic result is achieved. Generally, the dose is sufficient to treat or ameliorate symptoms or signs of a disease without producing unacceptable toxicity to the patient.

In one specific, non-limiting example, a unit dosage for intravenous or intramuscular administration of an ERRγ agonist includes at least 0.5 μg agonist per dose, such as at least 5 μg agonist per dose, at least 50 μg agonist per dose, or at least 500 μg agonist per dose. In some examples, doses are administered three-times in one week.

In one specific, non-limiting example, an ERRγ agonist daily dosage is from about 0.01 milligram to about 500 milligram per kilogram of animal body weight, for example given as a single daily dose or in divided doses two to four times a day, or in sustained release form. For most large mammals, the total daily dosage is from about 0.01 milligrams to about 100 milligrams per kilogram of body weight, such as from about 0.5 milligram to about 100 milligrams per kilogram of body weight, which can be administered in divided doses 2 to 4 times a day in unit dosage form containing for example from about 10 to about 100 mg of the compound in sustained release form. In one example, the daily oral dosage in humans is between 1 mg and 1 g, such as between 10 mg and 500 mg, 10 mg and 200 mg, such as 10 mg. The dosage regimen may be adjusted within this range or even outside of this range to provide the optimal therapeutic response. Oral administration of an ERRγ agonist can be carried out using tablets or capsules, such as about 10 mg to about 500 mg of the ERRγ agonist. Exemplary doses in tablets include 0.1 mg, 0.2 mg, 0.25 mg, 0.5 mg, 1 mg, 2 mg, 5 mg, 10 mg, 25 mg, 50 mg, 100 mg, 250 mg, and 500 mg of the ERRγ agonist. Other oral forms can also have the same dosages (e.g., capsules). In one example, a dose of an ERRγ agonist administered parenterally is at least 10 mg, such as 10 to 500 mg or 10 to 200 mg of the ERRγ agonist.

In one specific, non-limiting example, a unit dosage for oral administration (such as a table or capsule), or for oral intravenous or intramuscular administration, of an ERRγ protein includes about 1 μg to 1000 mg of ERRγ protein per dose, such as 1 μg to 100 μg ERRγ protein per dose, 1 μg to 500 μg ERRγ protein per dose, 1 μg to 1 mg ERRγ protein per dose, 1 mg to 1000 mg ERRγ protein per dose, or 10 mg to 100 mg ERRγ protein per dose. In some examples, doses are administered at least three-times in one week.

In one specific, non-limiting example, a unit dosage for administration of an ERRγ nucleic acid (such as injection, gene gun, pneumatic injection, or topical) includes at least 10 ng, at least 100 ng, at least 1 μg, at least 10 μg, at least 100 μg, or at least 500 μg nucleic acid per dose. Saline injections can use amounts of DNA, such as from 10 μg-1 mg, whereas gene gun deliveries can require 100 to 1000 times less DNA than intramuscular saline injection (such as 0.2 μg-20 μg). These amounts can vary from species to species, with mice, for example, requiring approximately 10 times less DNA than primates. Saline injections may require more DNA because the DNA is delivered to the extracellular spaces of a tissue (e.g., muscle or fat), where it has to overcome physical barriers before it is taken up by the cells, while gene gun deliveries bombard DNA directly into the cells.

In one specific, non-limiting example, a unit dosage for intravenous or intramuscular administration of a viral vector that encodes ERRγ includes at least 1×10⁸ viral particles per dose, such as at least 1×10⁹ viral particles per dose, at least 1×10¹⁰ viral particles per dose, or at least 1×10¹¹ viral particles per dose.

Actual methods for preparing administrable compositions will be known or apparent to those skilled in the art and are described in more detail in such publications as Remington's Pharmaceutical Science, 19th ed., Mack Publishing Company, Easton, Pa. (1995).

Agents that increase ERRγ activity (such as a ERRγ protein or agonist) can be provided in lyophilized form and rehydrated with sterile water before administration, although they are also provided in sterile solutions of known concentration. The resulting solution can then added to an infusion bag containing 0.9% sodium chloride, USP, and can be administered in some examples at a dosage of from 1 to 300 mg/kg of body weight. Considerable experience is available in the art in the administration of proteins or nucleic acids. Such molecules can be administered by slow infusion, rather than in an intravenous push or bolus. In one example, a higher loading dose is administered, with subsequent, maintenance doses being administered at a lower level.

The agents that increase ERRγ can be administered to humans or other mammal using routine modes of administration, such as topically, orally, intravascularly such as intravenously, intramuscularly, intraperitoneally, intranasally, intradermally, intrathecally, subcutaneously, intracraneally, via inhalation or via suppository. The particular mode of administration and the dosage regimen will be selected by the attending clinician, taking into account the particulars of the case (for example the subject, the disease, the disease state involved, and whether the treatment is prophylactic).

Controlled release parenteral formulations of agents that increase ERRγ activity can be made as implants, oily injections, or as particulate systems. For a broad overview of protein delivery systems (see Banga, A.J., Therapeutic Peptides and Proteins: Formulation, Processing, and Delivery Systems, Technomic Publishing Company, Inc., Lancaster, Pa., 1995). Particulate systems include microspheres, microparticles, microcapsules, nanocapsules, nanospheres, and nanoparticles. Microcapsules contain the therapeutic protein as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly (see Kreuter, J., Colloidal Drug Delivery Systems, J. Kreuter, ed., Marcel Dekker, Inc., New York, N.Y., pp. 219-342, 1994; Tice & Tabibi, Treatise on Controlled Drug Delivery, A. Kydonieus, ed., Marcel Dekker, Inc. New York, N.Y., pp. 315-339, 1992).

Polymers can be used for ion-controlled release. Various degradable and nondegradable polymeric matrices for use in controlled drug delivery are known in the art (Langer, R., Accounts Chem. Res. 26:537, 1993). For example, the block copolymer, polaxamer 407 exists as a viscous yet mobile liquid at low temperatures but forms a semisolid gel at body temperature. It has shown to be an effective vehicle for formulation and sustained delivery of recombinant interleukin-2 and urease (Johnston et al., Pharm. Res. 9:425, 1992; and Pec et al., J. Parent. Sci. Tech. 44:58, 1990). Alternatively, hydroxyapatite has been used as a microcarrier for controlled release of proteins (Ijntema et al., Int. J. Pharm. 112:215, 1994). In yet another aspect, liposomes are used for controlled release as well as drug targeting of the lipid-capsulated drug (Betageri, et al., Liposome Drug Delivery Systems, Technomic Publishing Co., Inc., Lancaster, Pa., 1993). Numerous additional systems for controlled delivery of therapeutic proteins are known (see, for example, U.S. Pat. Nos. 5,055,303, 5,188,837, 4,235,871, 4,501,728, 4,837,028 4,957,735 and 5,019,369, 5,055,303; 5,514,670; 5,413,797; 5,268,164; 5,004,697; 4,902,505; 5,506,206, 5,271,961; 5,254,342 and 5,534,496).

Site-specific administration of the agents that increase ERRγ activity can be used, for instance by applying the agent to a region of the body in need of treatment, such as the brain, adipose tissue, particular muscle, or kidney. In some embodiments, sustained release of the pharmaceutical preparation that includes a therapeutically effective amount of the one or more agents that increase ERRγ activity may be beneficial.

The present disclosure also includes combinations of one or more agents that increase ERRγ activity with one or more other agents useful in the treatment of a thermogenesis disorder. In one example, one or more beta adrenergic agonists are administered in combination with one or more agents that increase ERRγ activity. The term “administration in combination” or “co-administration” refers to both concurrent and sequential administration of the active agents. Examples of beta adrenergic agonists that can be used include, but are not limited to, a beta-adrenergic agonist (e.g., beta-2 or beta-3 agonist), and compounds that increase epinephrine secretion, such as phentermine.

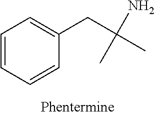

Beta₂-adrenergic agonists (β2 agonists) are a class of compounds that act on the beta₂-adrenergic receptor. Examples of β2 agonists that can be used with the disclosed methods include but are not limited to: a short acting β2 agonist, such as salbutamol (aka albuterol), levosalbutamol (aka levalbuterol), terbutaline, pirbuterol, procaterol, clenbuterol, metaproterenol, fenoterol, bitolterol mesylate, ritodrine, and isoprenaline; a long-acting β2 agonist such as salmeterol, formoterol, bambuterol, clenbuterol, or olodaterol; or an ultra-long-acting β2 agonist such as indacaterol, and combinations thereof. Additional non-limiting examples of β2 agonists include but are not limited to epinephrine, norepinephrine, isoproterenol, GSK-159797, GSK-597901, GSK-159802, GSK-642444, GSK-678007, and combinations thereof. In some examples, a β2 agonist is administered using an inhaler, such as a metered-dose inhaler, which aerosolizes the drug, or dry powder, which can be inhaled. In some examples, a β2 agonist is administered in a solution form for nebulization. In some examples, a β2 agonist is administered orally or intravenously (or other form of injection).

In some examples, the beta adrenergic agonist is a β3 agonist, such as amibegron (SR-58611A), CL-316,243, L-742,791, L-796,568, LY-368,842, mirabegron (YM-178), Ro40-2148, solabegron (GW-427,353), BRL 37344, ICI 215,001, L-755,507, ZD 2079, ZD 7114, or combinations thereof.

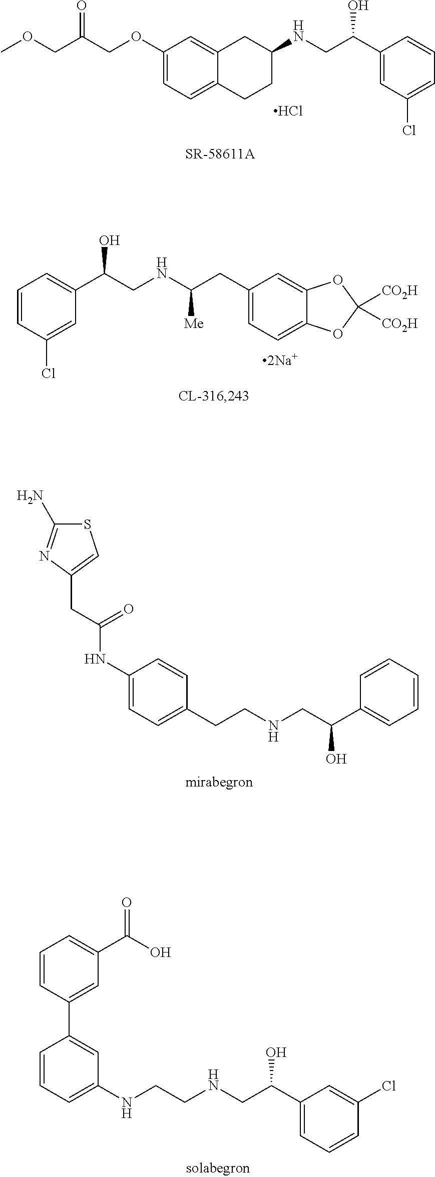 [SMILES: COCC(=O)COc1ccc2c(c1)C[C@@H](NC[C@H](O)c1cccc(Cl)c1)CC2 ; Cl ; "SR-58611A" ; C[C@H](Cc1ccc2c(c1)OC(C(=O)O)(C(=O)O)O2)NC[C@H](O)c1cccc(Cl)c1 ; Nc1nc(CC(=O)Nc2ccc(CCNC[C@H](O)c3ccccc3)cc2)cs1 ; O=C(O)c1cccc(-c2cccc(NCCNC[C@H](O)c3cccc(Cl)c3)c2)c1]

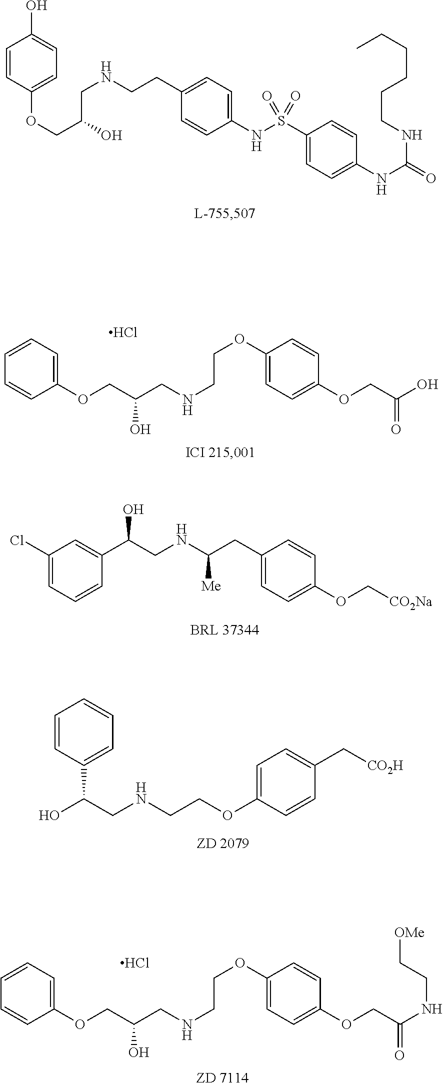

In some examples, a β3 agonist is administered orally or intravenously (or other form of injection). In some examples, a β3 agonist is administered using an inhaler, such as a metered-dose inhaler, which aerosolizes the drug, or dry powder, which can be inhaled.

### Compositions

Also provided are compositions that include one or more agents that increase ERRγ activity and one or more beta adrenergic agonists. The agent that increases ERRγ activity can be a nucleotide sequence encoding for an ERRγ protein, an agent which enhances ERRγ expression, a substantially purified ERRγ protein, an ERRγ agonist, or combinations thereof. Any of the specific agents that increase ERRγ activity disclosed herein can be present in the composition. The beta adrenergic agonist can be any beta adrenergic agonist disclosed herein, such as a β2 agonist or β3 agonist provided herein. In some examples, the composition also includes a pharmaceutically acceptable carrier.

### Example 1

### ERRγ is Highly Expressed in Mature Brown Adipocytes but is not Induced by Chronic Cold Exposure

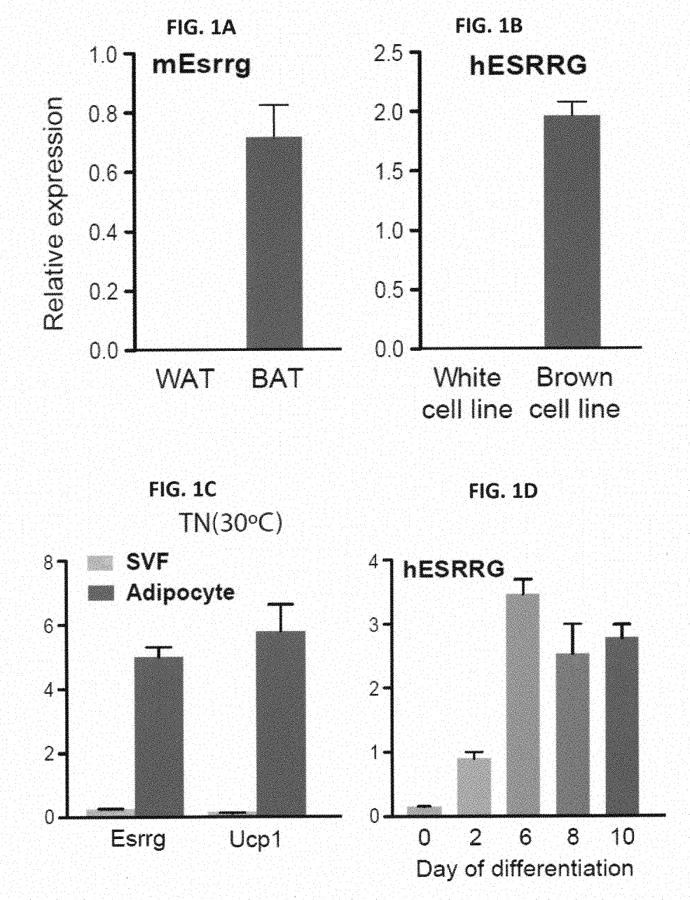

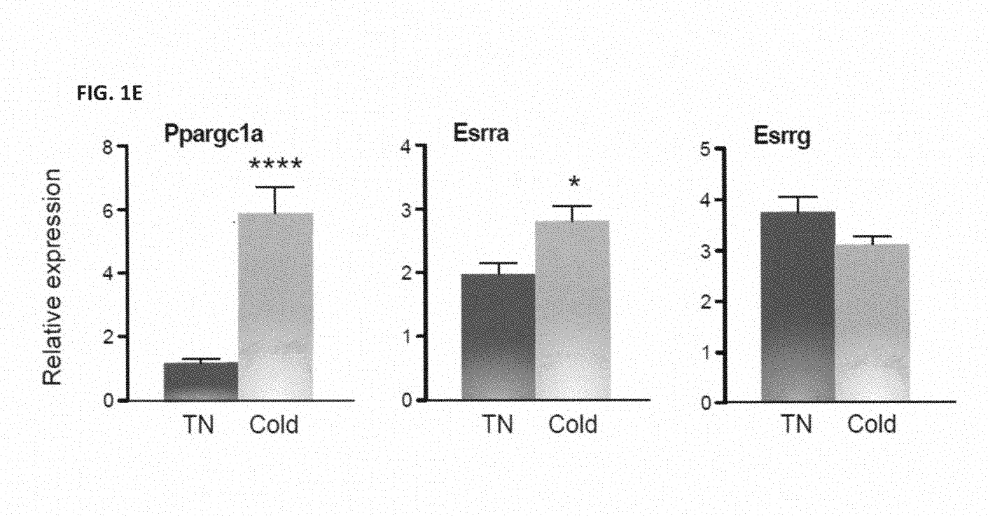

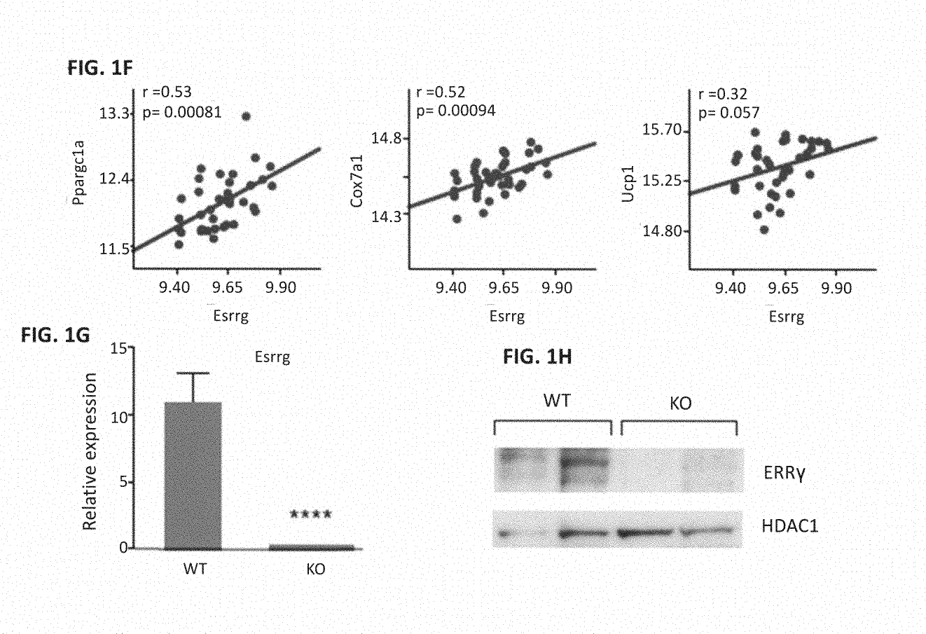

ERRγ mRNA levels were highly expressed in BAT versus WAT in both mouse adipose tissue as well as in human adipocyte cells lines (FIGS. 1A and B) (23). ERRγ is also more highly expressed in the mature adipocyte fraction compared to the stromal vascular fraction (SVF), indicating that it has a role in mature brown adipocyte function (FIG. 1C). Utilizing the PAZ-6 human brown adipose cell line (23), it was observed that ERRγ is induced late during differentiation, further supporting a role for ERRγ in mature brown adipocyte function (FIG. 1D). While ERRγ is highly expressed in BAT versus WAT, ERRγ is not induced during chronic cold exposure, like other transcription factors known to control BAT metabolism such as ERRγ and PGC1α (FIG. 1E), indicating that it may play a role in the basal (thermoneutral) state (12).

Using genetically diverse strains of mice from the BXD genetic reference population, it was observed that expression of ERRγ in BAT was positively correlated with important BAT genes such as PGC1a, Cox7a1 and UCP-1, further indicating that ERRγ plays a critical role in BAT (FIG. 1F).

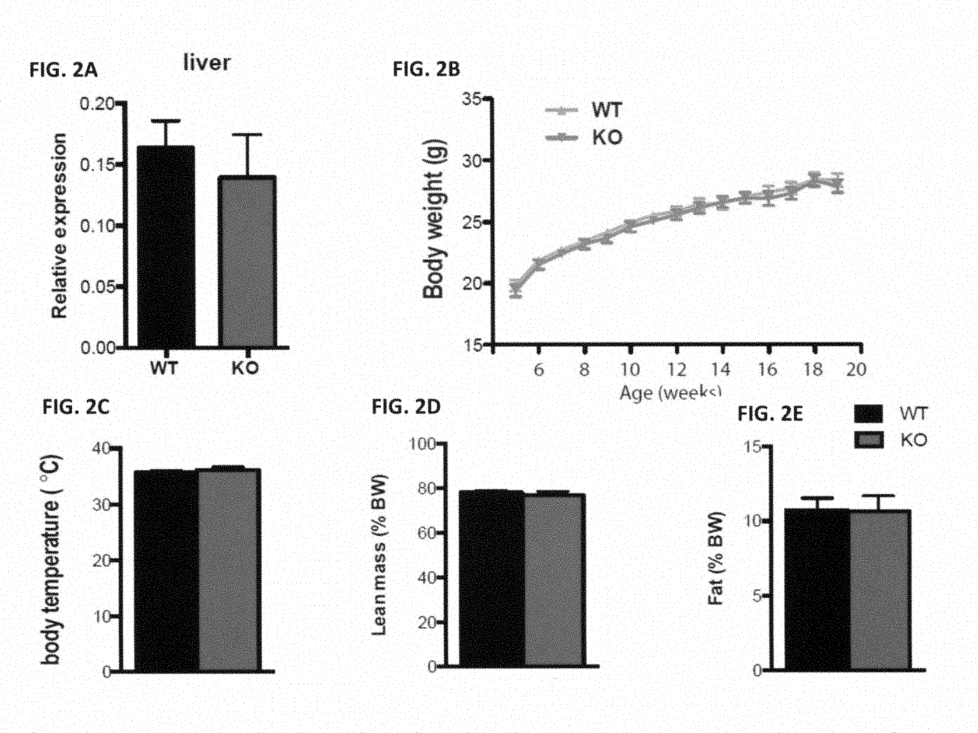

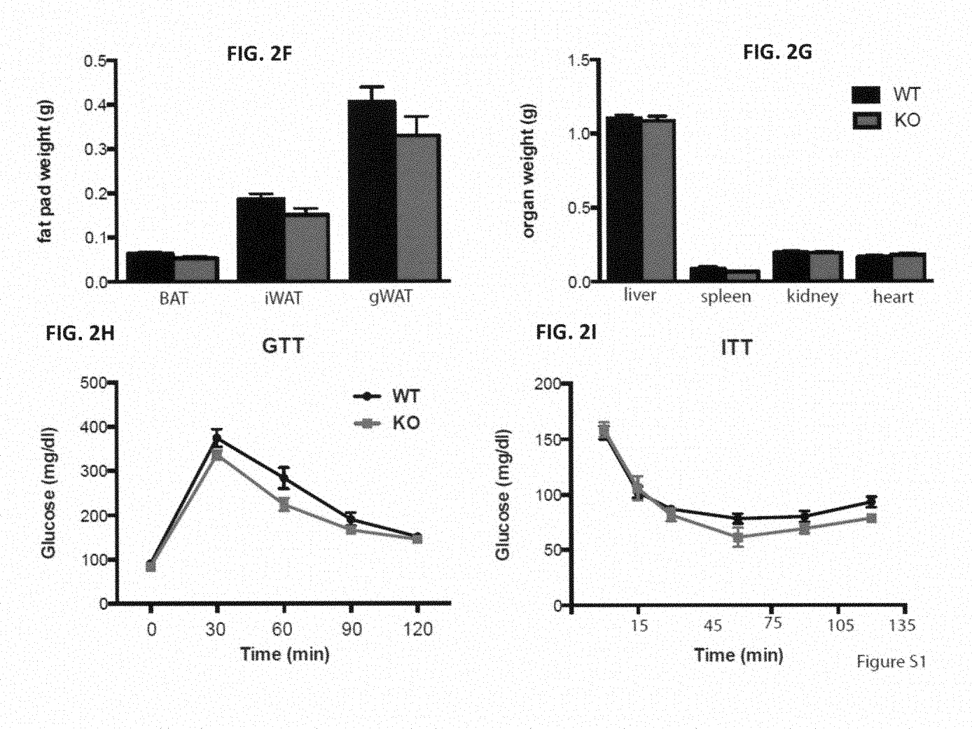

To investigate the role of ERRγ in basal BAT function, ERRγ adipose conditional KO mice (ERRγ-ASKO) were generated by crossing ERRγ flox/flox mice with adiponectin-Cre mice. ERRγ was sufficiently deleted at the mRNA level in BAT and not in other tissues such as the liver (FIGS. 1G and 2A). Similarly, while ERRγ protein levels were easily detectable in BAT of control flox/flox mice, they were absent in BAT from ERRγ-ASKO mice (FIG. 1H). ERRγ-ASKO mice are born at a normal Mendelian ratio and under standard animal housing condition exhibit no obvious phenotypic abnormalities (FIGS. 2B-2I).

### Example 2

### ERR7 is Required to Maintain Expression of Thermogenic Genes Under Basal Conditions

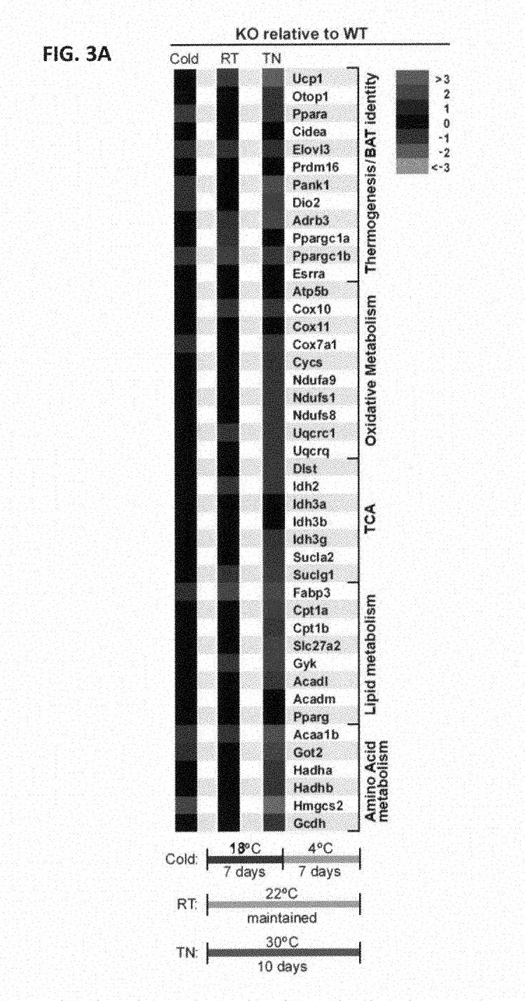

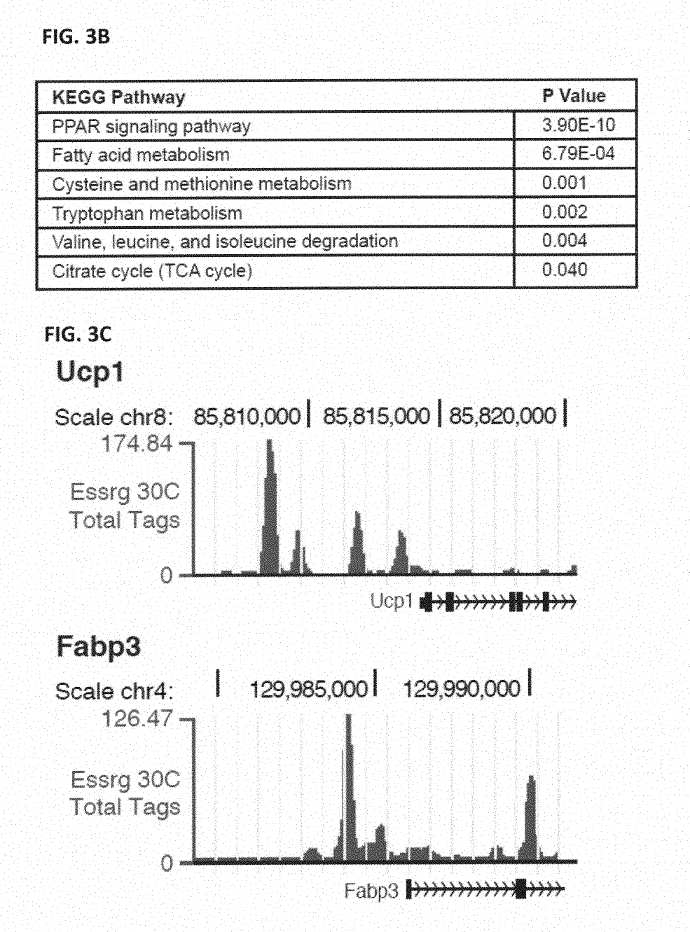

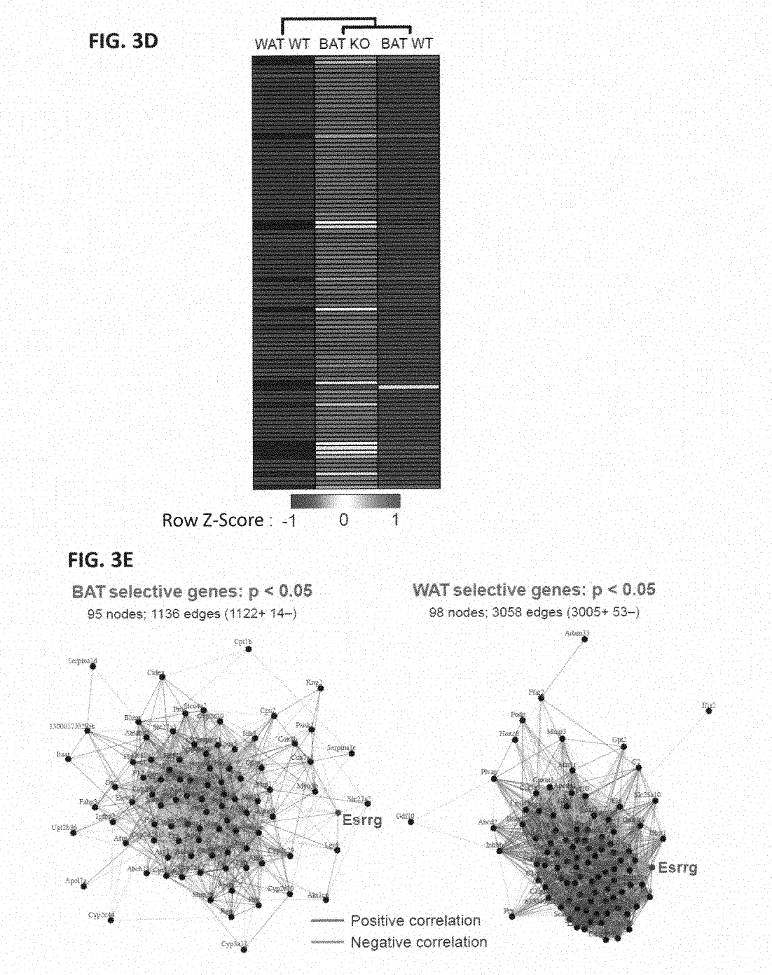

The function of ERRγ in BAT metabolism was determined by housing ERRγ-ASKO mice and control flox/flox littermates under chronic cold acclimated conditions (4° C.), mild cold stress (room temperature (22° C.)) or thermoneutrality (30° C.) (FIG. 3A lower panel) and compared their BAT gene signatures by RNA-sequencing (RNA-Seq). Under chronic cold conditions, the expression of 293 genes was significantly changed (142 downregulated and 149 upregulated). Under moderate cold (22° C.) 71 genes changed (37 downregulated and 34 upregulated). At thermoneutrality, there were the most gene expression changes, with 556 genes significantly changed (317 downregulated and 239 upregulated). The expression levels of several genes important for BAT identity were determined, including genes important in thermogenesis, electron transport, TCA, lipid metabolism and amino acid metabolism (FIG. 3A, upper panel). Under severe and mild cold conditions, minimal changes in the expression of these genes was observed. However, at thermoneutrality, marked downregulation of many BAT signature genes in ERRγ-ASKO BAT compared to flox/flox mice was observed (FIG. 3A). In this regard, pathway analysis of all the downregulated genes in ERRγ-ASKO BAT, under thermoneutral conditions, revealed that the predominant altered pathways were metabolic (such as those involved in PPAR signaling, fatty acid and amino acid metabolism, as well as the TCA cycle) (FIG. 3B).

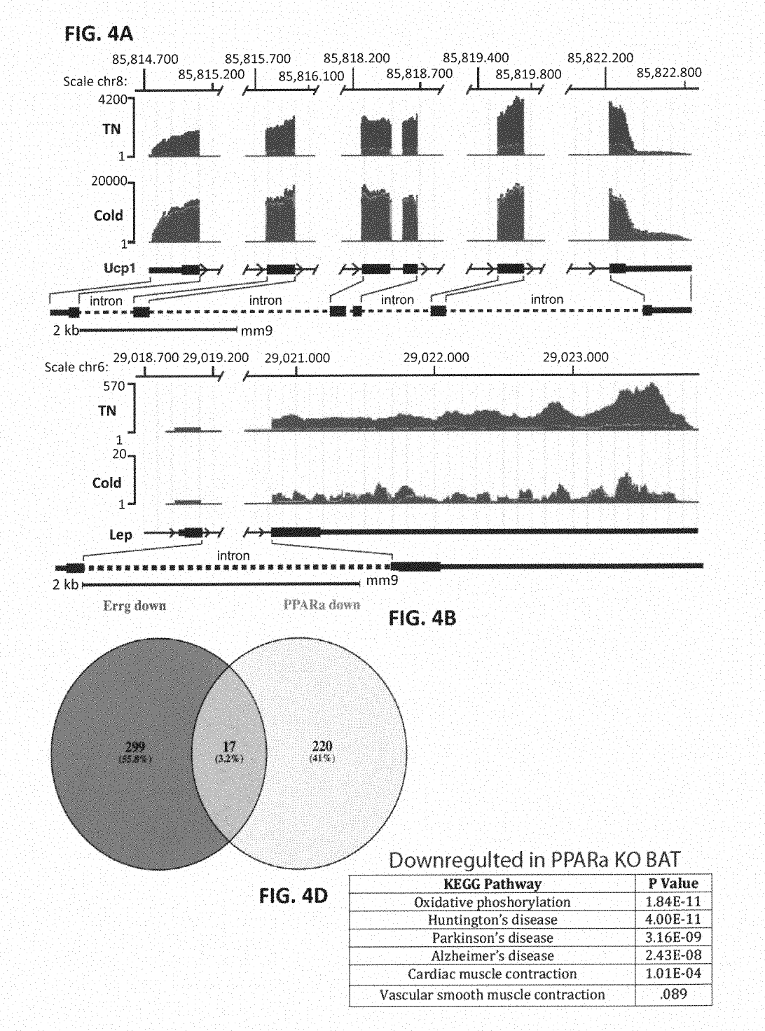

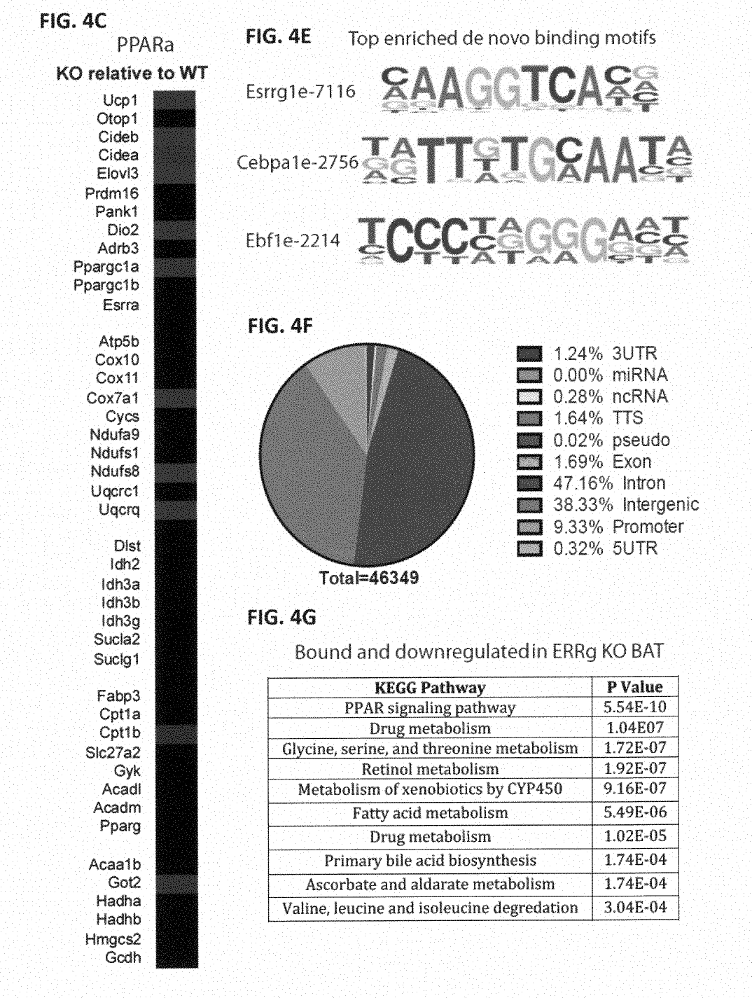

Downregulation of PPARa expression was detected in BAT from ERRγ ASKO mice during thermoneutrality (FIG. 3A). PPARa regulates the expression of genes involved in fatty acid utilization and thermogenesis in BAT. Therefore, some of the downregulated BAT signature genes in ERRγ ASKO mice could be secondary to decreased PPARα expression. To rule out an indirect effect of ERRγ, RNA-Seq analysis of BAT from PPARα null mice, under thermoneutral conditions, was examined. Pathway analysis as well as a heatmap of key BAT genes from BAT of PPARα null mice and ERRγ ASKO mice revealed that these two nuclear receptors control distinct gene sets (FIGS. 3A-3B and 4B-4D). Comparison of all significantly downregulated genes from BAT in PPARα null mice and ERRγ ASKO mice showed that there was only 3% overlap in downregulated genes (FIG. 4B). Thus, the changes in gene signature in ERRγ deficiency during thermoneurality is not via PPARα and probably due to direct binding of ERRγ.

To further demonstrate that ERRγ directly controls the expression of metabolic genes in BAT, chromatin immunoprecipitation was performed followed by deep sequencing (ChIP-Seq) to determine the genome wide binding sites of ERRγ in BAT of wild type mice acclimated to thermoneutrality. The majority of binding sites were enriched in ERRγ motifs, indicating direct DNA binding (FIG. 4E). Similar to many transcription factors in ChIP-Seq analysis, most binding sites were in intergenic and intronic regions (FIG. 4F). ERRγ directly bound to the proximal promoters of key BAT genes, such as Ucp1 and Fabp3, which were downregulated in ERRγ-ASKO BAT by RNA-Seq. Pathway analysis of all bound and downregulated genes revealed that the most predominantly affected pathways were metabolic, indicating that ERRγ binds to key BAT metabolic genes to maintain their expression (FIG. 4G).

As shown above, RNA-Seq revealed that UCP-1 expression, a hallmark of functional BAT, was decreased by 65% under thermoneutral conditions. In contrast, leptin, which is positively correlated with a hypofunctional state of BAT, was induced 2.6-fold in BAT of ERRγ-ASKO mice only at thermoneutrality (27) (FIG. 4A). To determine if BAT from ERRγ-ASKO mice had lost its unique characteristics, and therefore had become more “WAT-like”, expression of the top 100 BAT versus WAT selective genes under basal conditions in ERRγ KO BAT was examined. Remarkably, expression of almost all these BAT-selective genes was decreased in ERRγ KO BAT, such that it resembled a more WAT-like gene signature (FIG. 3D). Furthermore, in ChIP-Seq experiments, 50% of these BAT-selective genes were bound by ERRγ. To investigate if ERRγ expression correlates with BAT versus WAT-selective gene expression, the expression of ERRγ in both BAT and WAT selective gene networks of BAT across BAT from BXD genetically diverse strains of mice was examined (28). A relationship of ERRγ was detected with both BAT and WAT selective gene networks across BAT of BXD mice. However, while ERRγ was positively associated with many BAT selective genes, its expression exhibited a strong negative correlation with WAT selective genes (FIG. 3E). Taken together, these findings show that ERRγ is required for maintaining the expression of key BAT genes in the basal state and potentially suppresses WAT selective genes.

### Example 3

### ERRγ-ASKO Mice Exhibit a BAT to WAT Phenotype with Impaired Fatty Acid Utilization

Since the major differences in ERRγ-ASKO BAT relative to control flox/flox mice were observed at thermoneutrality, and to eliminate any compensatory influence of the sympathetic nervous system, the remainder of the studies were performed under thermoneutral conditions.

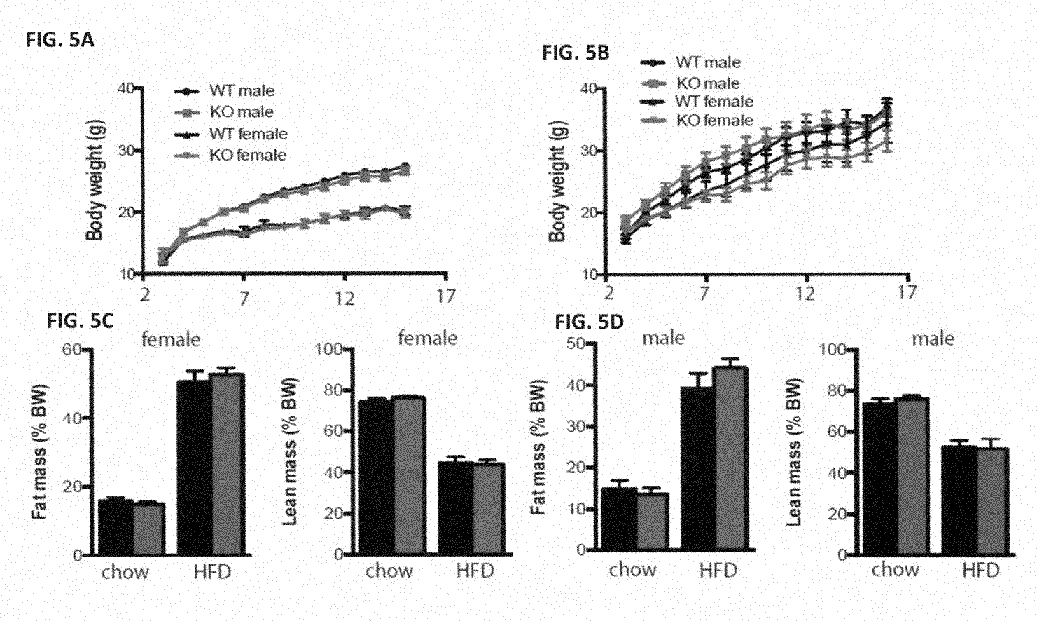

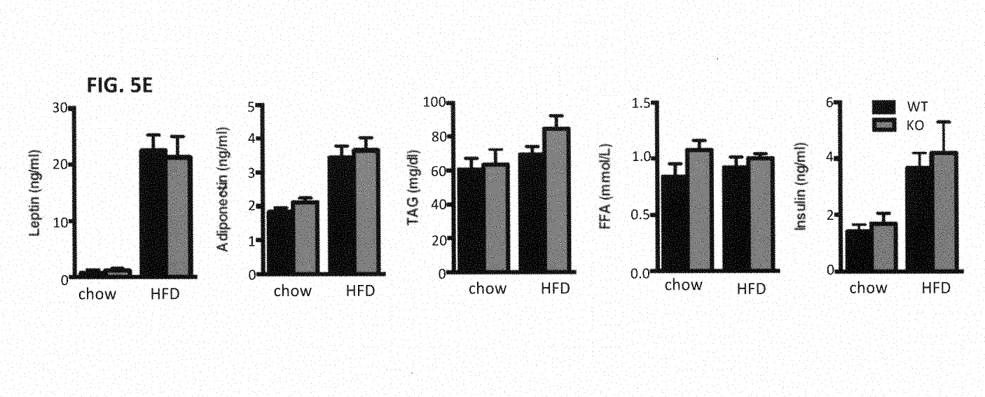

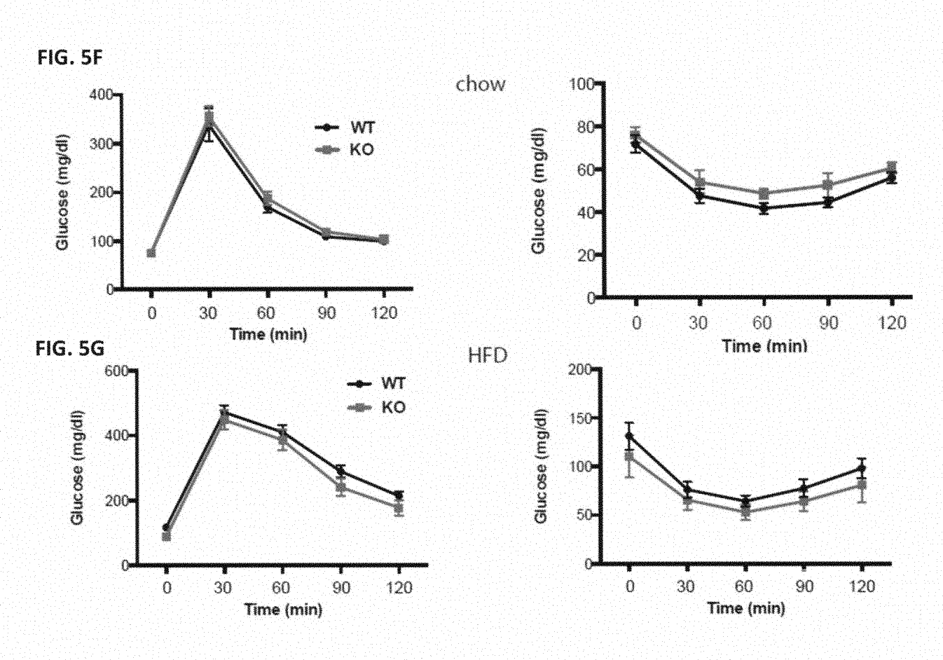

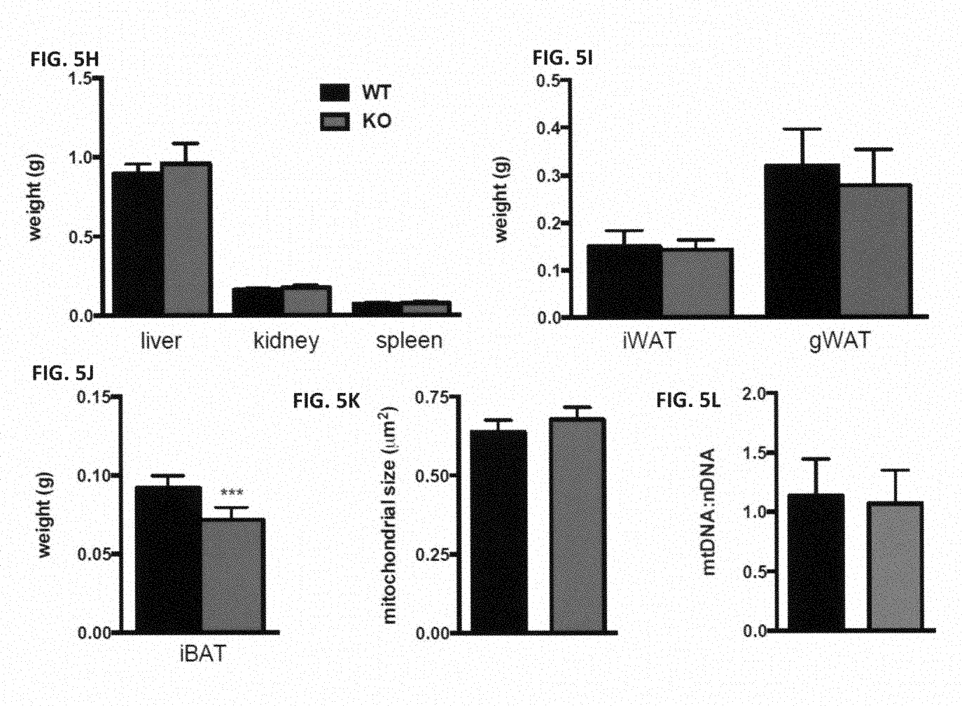

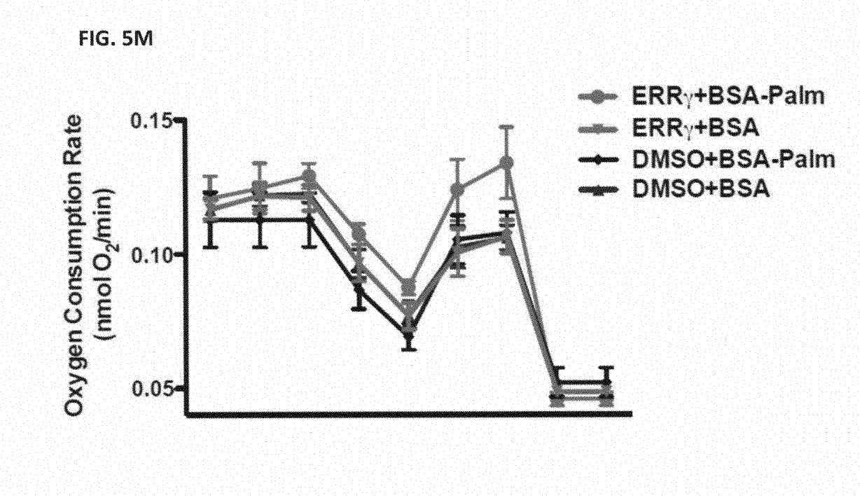

Both ERRγ-ASKO mice and control flox/flox mice were placed on a standard chow or high fat diet (HFD) and their body weights monitored for 16 weeks. Both groups of mice gained similar weights on both chow and HFD (FIGS. 5A-5B). In this regard, there was no difference in body composition, serum parameters or insulin and glucose tolerance between the two groups of mice (FIGS. 5C-5G). There were no apparent differences in gross appearance and weights of major metabolic organs in ERRγ-ASKO mice (FIGS. 5H-5J).

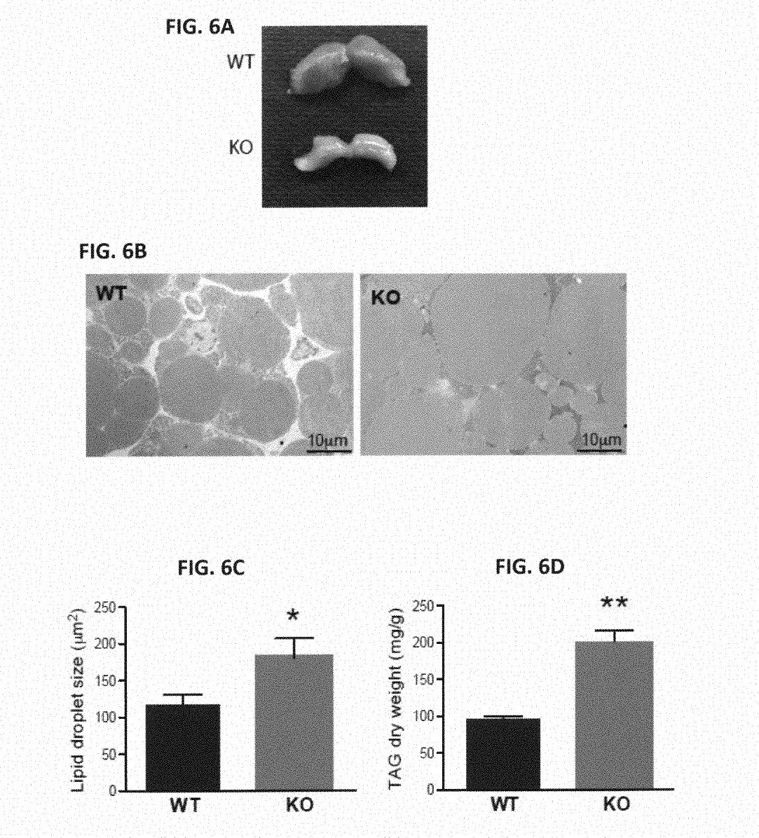

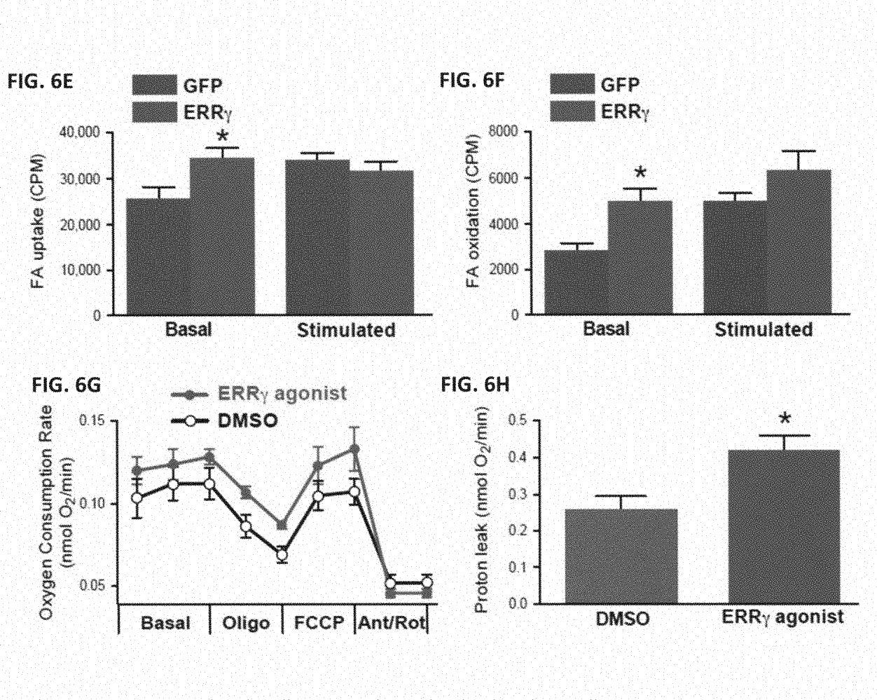

However, BAT from ERRγ-ASKO mice appeared markedly different from that of control flox/flox mice (FIGS. 5J and 6A). ERRγ-ASKO BAT was smaller and paler, taking on a more “WAT-like” appearance. Transmission electron microscopy revealed that while mitochondria number and size were unchanged (FIGS. 5K-5L), the size of lipid droplets were increased in ERRγ-ASKO BAT, resembling WAT (FIGS. 6B-6C). In this regard, TAG levels in BAT were also increased (FIG. 6D). Since results from the RNA-Seq analysis showed impaired expression of genes involved in fatty acid uptake and oxidation in ERRγ-ASKO BAT, it was hypothesized that the increased lipid accumulation could be partially due to an impairment in these processes. Therefore, ERRγ was overexpressed in differentiated PAZ-6 human brown adipocytes and fatty acid uptake and oxidation measured. Indeed, both fatty acid uptake and oxidation were increased under basal conditions in cells overexpressing ERRγ (FIGS. 6E and 6F). Notably, this increase in fatty acid uptake and oxidation was not observed under stimulated conditions (treatment with the beta adrenergic agonist norepinephrine), consistent with the finding that fatty acid utilization genes were only changed under basal conditions in ERRγ-ASKO BAT. Oxygen consumption rate and proton leak or uncoupling was higher in differentiated PAZ-6 treated with an ERRγ agonist (FIGS. 6G-6H). Interestingly, this increase only occurred in the presence of fatty acids (FIG. 5M), suggesting that the effect of activation of the receptor was restricted to increased fat oxidation.

### Example 4

### ERR7 is Required for Survival During Acute Cold Exposure

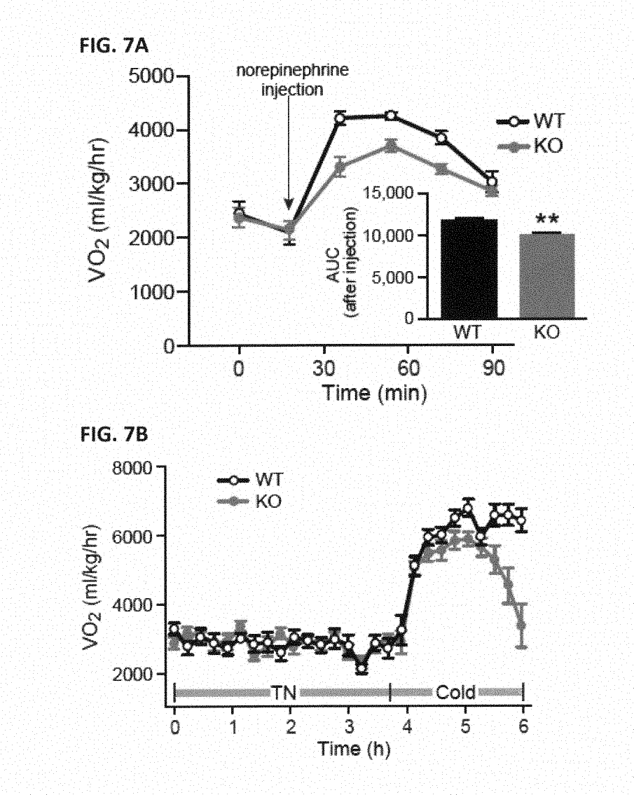

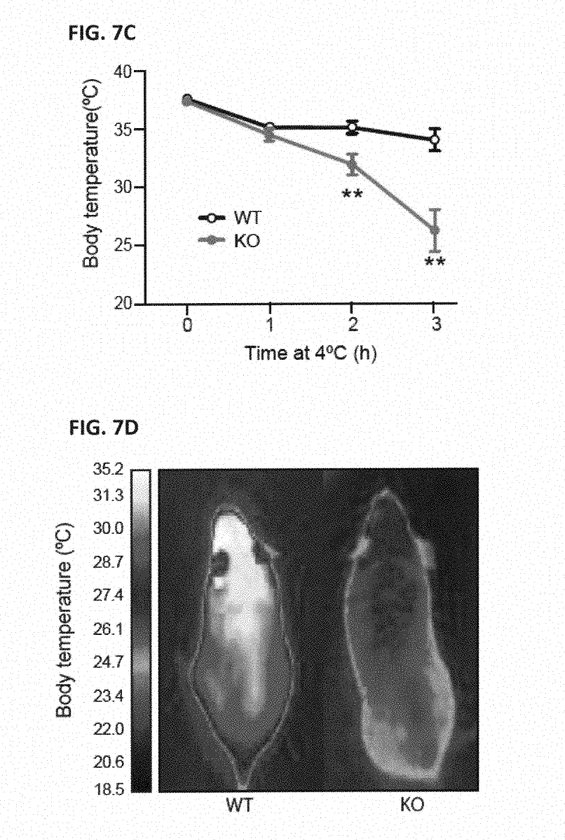

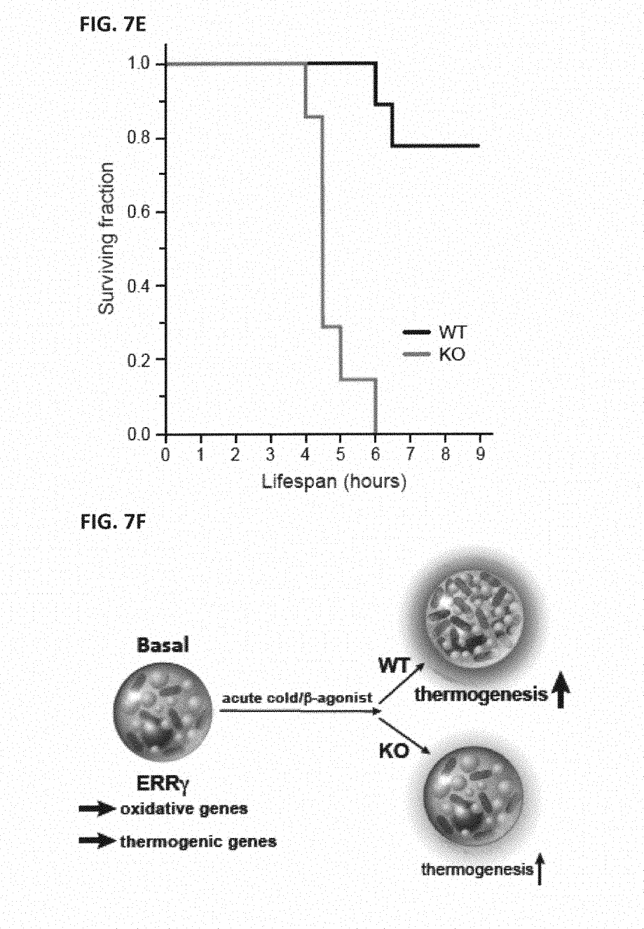

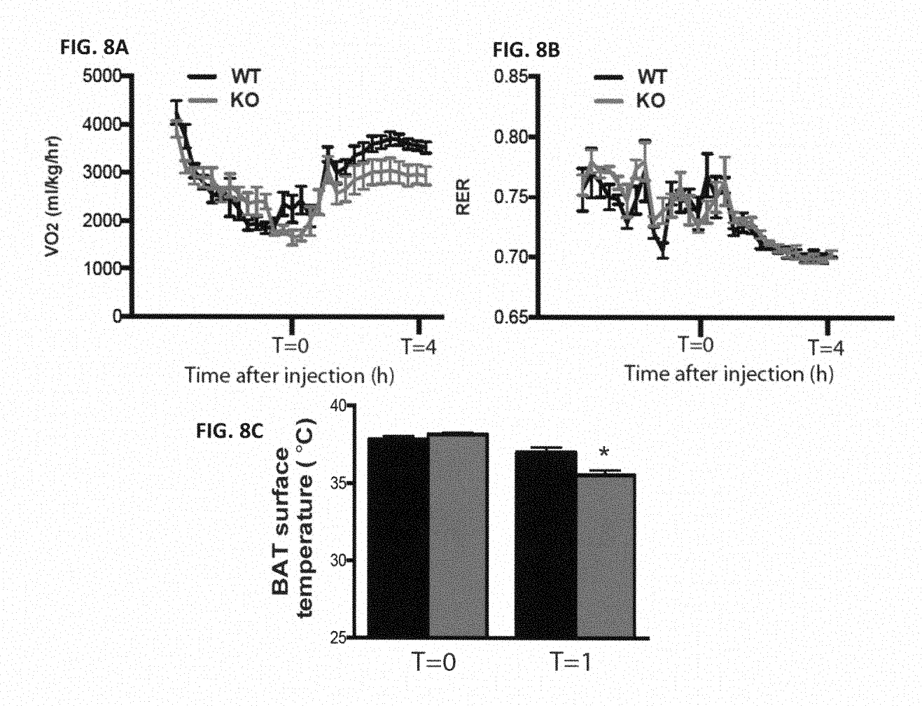

To determine the physiological consequence of loss of ERRγ in BAT, the thermogenic capacity of BAT from ERRγ-ASKO mice was tested under basal conditions by injecting norepinephrine (NE) and monitoring oxygen consumption (VO₂). While control flox/flox mice increased their metabolic rate upon NE injection, this response was severely blunted in ERRγ-ASKO mice, indicating an impaired thermogenic capacity at thermoneutrality (FIG. 7A). A similar blunted response to the β3 agonist CL316243 was observed (FIGS. 8A-8B).

Since ERRγ-ASKO mice exhibit an impaired thermogenic capacity under basal conditions, it was hypothesized that they would be unable to properly induce thermogenesis when challenged with acute cold exposure. The oxygen consumption rate of ERRγ-ASKO mice and flox/flox littermates was measured upon exposure to acute cold. There was no difference in oxygen consumption under basal conditions between ERRγ-ASKO mice and flox/flox control mice (FIG. 7B). However, while flox/flox mice markedly increased their oxygen consumption rate upon exposure to cold, ERRγ-ASKO mice failed to maintain this high metabolic rate, as it plummeted upon continued exposure to cold (FIG. 7B). Accordingly, while flox/flox control mice maintained their body temperature upon exposure to acute cold, after only 3 hours ERRγ-ASKO mice were reaching life-threatening hypothermia (FIG. 7C). Thermographic surface measurements revealed that BAT surface temperature was indeed decreased in ERRγ-ASKO mice upon exposure to cold (FIGS. 7D and 8C). This inability of ERRγ-ASKO mice to maintain body temperature was associated with impaired survival. After six hours of acute cold exposure, one hundred percent of ERRγ-ASKO mice had died compared to a twenty percent mortality of control flox/flox mice after 9 hours cold exposure (FIG. 7E).

Thus, ERRγ is required for basal BAT function by maintaining the expression of genes involved in thermogenesis and fatty acid utilization. In contrast, during cold stimulation or β agonist treatment, BAT becomes activated. Other transcription factors, such as ERRα and PGC1α, are induced in the brown adipocyte to further promote the expression of thermogenic and fatty acid oxidation genes and enable an animal to adapt to cold conditions (FIG. 7F). Nevertheless, if BAT is not properly primed for thermogenesis in the basal state, as shown here with loss of ERRγ, this leads to a whitening of BAT and the inability to survive an acute cold challenge.

### Example 5

### Experimental Procedures

The following methods were used for the studies described in Examples 1-4.

### Animal Studies

ERRγ flox/flox mice were crossed with adiponectin-Cre mice (Jackson Laboratory) to generate ERRγ-ASKO mice. ERRγ-ASKO mice and flox/flox control littermates received a standard chow diet (MI laboratory rodent diet 5001, Harlan Teklad) or high fat (60%) diet (F3282, Bio-Serv) and water ad libitum. All mice used for studies were male unless otherwise noted. Mice were housed at thermoneutrality (30° C.) unless otherwise indicated. PPARα null mice were purchased from Jackson laboratory.

### RNA Extraction and Gene Expression Analysis

Total RNA was isolated from mouse tissue and cells using TRIzol reagent (Invitrogen). Complementary DNA was synthesized from 1 mg of DNase-treated total RNA using SuperScript II reverse transcriptase (Invitrogen). mRNA levels were quantified by qPCR with SYBR Green (Invitrogen). Samples were run in technical triplicates and relative mRNA levels were calculated by using the standard curve methodology and normalized against cyclophilin (mouse) 36B4 (human) mRNA levels in the same samples.

### RNA Sequencing and Analysis

10-week-old male chow-fed WT and KO mice were housed at 30° C. for 10 days, or at 18° C. for one week followed by 4° C. for an additional week, for warm and cold acclimation, respectively (Lim et al., Nature protocols 7:606-615, 2012). Mice were sacrificed at 2 pm in the ad libitum state. RNA was isolated from mouse tissues, with biological triplicates for all conditions. Total RNA was extracted using Trizol (Invitrogen) and the RNeasy mini kit (Qiagen). RNA purity and integrity were confirmed using an Agilent Bioanalyzer. Libraries were prepared from 100 ng total RNA (TrueSeq v2, Illumina) and single-ended sequencing performed on the Illumina HiSeq 2500, using bar-coded multiplexing and a 100 bp read length, yielding a median of 34.1M reads per sample. Read alignment and junction finding was accomplished using STAR (Dobin, et al., Bioinformatics 29:15-21, 2013) and differential gene expression with Cuffdiff 2 (Trapnell et al., Nat Biotechnol 31:46-53, 2013), utilizing UCSC mm9 as the reference sequence.

### ChIP Sequencing and Analysis

A total of 20 mice pre-conditioned in 30 C were sacrificed and their brown adipose tissues (BAT) were dissected and pooled into 10 cm tissue culture dish with PBS. BAT was minced into ˜1 mm×1 mm fine fragments. Disuccinimidyl glutarate (DSG) was added directly into PBS to a final concentration of 2 mM and tissues were allowed to fix at room temperature for 30 minutes. Immediately following DSG fixation, Paraformaldehyde was added to 1% final concentration and tissues were cross-linked an additional 10 minutes. Cross-linking was stopped by 1/20 volume of 2.5M glycine. Minced BAT was collected into 50 ml conical tubes and spun at 2500 rpm for 5 minutes at room temperature to remove cross-linking solution and cell debris at the bottom of the tube. The upper adipose tissue layer was washed with 10 ml ice cold PBS twice and spun at 2500 rpm at 4° C. for 5 minutes. 3 ml pre-chilled nuclei isolation buffer (20 mM Tris pH8, 85 mM KCl, 0.5% NP-40 and protease inhibitor cocktail) was added to BAT. Adipose tissue was disrupted by Teflon douncer at 4° C., after which cell homogenates were allowed to further swell in nuclei isolation buffer for 10 minutes on ice. Cell homogenates were filtered with 100 μm cell strainer to remove large debris. Adipose nuclei were pelleted by centrifugation at 2500 rpm for 5 minutes at 4° C. Nuclei were washed twice by nuclei isolation buffer. Isolated BAT nuclei were sonicated with a Biorupter in sonication buffer (1% SDS, 50 mM Tris pH 7.5, 10 mM EDTA and protease inhibitor cocktail) to isolate BAT chromatin. Sonicated BAT chromatin was diluted 10× in dilution buffer (16.7 mM Tris pH 8.0, 0.01% SDS, 1.1% Triton X-100, 1.2 mM EDTA, 167 mM NaCl and protease inhibitor). 10 μg of ERRγ antibody was used for immunoprecipitation overnight at 4° C. 50 μl of magnetic protein G beads was added to immunoprecipitation buffer for additional 2 hours of incubation at 4 degrees. Magnetic beads were collected and washed once by low salt wash buffer (1% Triton X-100, 0.1% SDS, 150 mM NaCl, 1 mM EDTA, 20 mM Tris pH 8.0 and 0.1% Na-deoxycholate), twice by high salt wash buffer (1% Triton X-100, 0.1% SDS, 500 mM NaCl, 1 mM EDTA, 20 mM Tris pH 8.0, and 0.1% Na-deoxycholate), once by LiCl wash buffer (250 mM LiCl, 0.5% NP-40, 1 mM EDTA, 20 mM Tris pH 8.0, and 0.5% Na-deoxycholate), and twice by TE wash buffer (10 mM Tris pH 8.0 and 1 mM EDTA). Chromatin immuno-complexes were eluted from the beads by successive treatments of 200 μl elution buffer (0.1M NaHCO₃ and 1% SDS) at room temperature with rotation for 15 minutes. Eluted chromatin was reverse cross-linked at 55 degrees with 0.2 mg/ml proteinase K overnight. DNA was purified by phenol/chloroform extraction and quantified by qubit fluorometer.

### Cold Exposure

Mice were acclimated to thermoneutrality for 10 days and then transferred to cold. Food was removed at the beginning of cold exposure.

### Cell Culture

PAZ-6 human brown adipocytes were differentiated as previously described (18). Human adipose derived stem cells were cultured as previously described.

### TAG Quantification

Total neutral lipids were extracted by the method of Folch (Folch et al., J. Biol. Chem. 226:297-509, 1957). Lipids were solubilized in 1% TritonX-100 and TAG was measured using Infinity Reagent (Thermo).

### Electron Microscopy

Freshly dissected brown fat was immersion fixed in ice-cold fixative consisting of 2.5% paraformaldehyde, 3% glutaraldehyde and 0.02% picric acid in 0.1M cacodylate buffer for 1 week at 4° C. The tissues were then buffer washed, post fixed in buffered 2% osmium tetroxide and subsequently dehydrated in graded ethanol series, transitioned in propylene oxide and embedded in Spurr resin (Electron Microscopy Sciences, Hatfield Pa.). Thick sections (1 μm) were cut, mounted on glass slides and stained in toluidine blue for general assessment in the light microscope. Subsequently, 70 nm thin sections were cut with a diamond knife (Diatome, Hatfield Pa.), mounted on copper slot grids coated with parlodion and stained with uranyl acetate and lead citrate for examination on a Philips CM100 electron microscope (PEI, Hillsboro, Oreg.). Images were documented using a Megaview III ccd camera (Olympus Soft Imaging Solutions GmbH, Münster, Germany).

### CLAMS

Oxygen consumption (VO₂) was measured using the Comprehensive Laboratory Animal Monitoring System (CLAMS; Columbus, Ohio). Data were normalized to body weights. Body temperatures were assessed using a RET-3 rectal probe for mice (Physitemp). CL316243 (Sigma) and norepinephrine were intraperitoneally injected into mice at 1 mg/kg body weight.

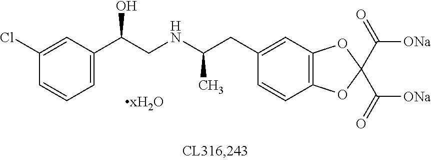

### Western Blot

Nuclear lysates (15 μg) were resolved using 10% SDS-PAGE, transferred to nitrocellulose membranes, and probed with anti-UCP-1 (Sigma), ERRγ, HDAC1 primary antibodies followed by horseradish peroxidase conjugated secondary antibody (Biorad). Blots were visualized using enhanced chemiluminescence substrate (PerkinElmer) and images were captured.

### XF Analysis

Bioenergetic profiles were analyzed using a XF24XF analyzer (Seahorse Bioscience) and the XF Palmitate-BSA FAO Substrate (Seahorse Bioscience).

### REFERENCES

1. Rosen and Spiegelman, Cell 156, 20-44 (2014).
2. Cannon and Nedergaard, Physiological reviews 84, 277-359 (2004).
3. Cinti, Disease models & mechanisms 5, 588-594 (2012).
4. Cypess et al., The New England journal of medicine 360, 1509-1517 (2009).
5. van Marken et al., The New England journal of medicine 360, 1500-1508 (2009).
6. Virtanen et al., Functional brown adipose tissue in healthy adults. The New England journal of medicine 360, 1518-1525 (2009).
7. Zingaretti et al., FASEB journal: official publication of the Federation of American Societies for Experimental Biology 23, 3113-3120 (2009).
8. Jespersen et al., Cell metabolism 17, 798-805 (2013).
9. Lidell et al., Nature medicine 19, 631-634 (2013).
10. Cypess et al., Nature medicine 19, 635-639 (2013).
11. Kajimura et al., Cell metabolism 22, 546-559 (2015).
12. Villena et al., Proceedings of the National Academy of Sciences of the United States of America 104, 1418-1423 (2007).
13. Dempersmier et al., Molecular cell 57, 235-246 (2015).
14. Narkar et al., Cell metabolism 13, 283-293 (2011).
15. Deblois and Giguere, Biochimica et biophysica acta 1812, 1032-1040 (2011).
16. Eichner and Giguere, Mitochondrion 11, 544-552 (2011).
17. Pei et al., Cell metabolism 21, 628-636 (2015).
18. Dufour et al., Cell metabolism 5, 345-356 (2007).
19. Kida et al., Cell stem cell 16, 547-555 (2015).
20. Wang et al., Molecular and cellular biology 35, 1281-1298 (2015).
21. Alaynick et al., Cell metabolism 6, 13-24 (2007).
22. Alaynick et al., Mol Endocrinol 24, 299-309 (2010).
23. Kazantzis et al., Frontiers in endocrinology 3, 13 (2012) 10.3389/fendo.2012.00013).
24. Wu et al., Cell 158, 1415-1430 (2014).
25. Dong et al., International journal of obesity, (2016).
26. Knoll et al., PloS one 8, e55884 (2013)10.1371/journal.pone.0055884).
27. Cinti et al., Endocrinology 138, 797-804 (1997).
28. Andreux et al., Cell 150, 1287-1299 (2012).
29. Harms and Seale, Nature medicine 19, 1252-1263 (2013).
30. Harms et al., Cell metabolism 19, 593-604 (2014).
31. Dixen et al., Obesity 21, 516-524 (2013).
32. Gantner et al., Proceedings of the National Academy of Sciences of the United States of America 111, 11870-11875 (2014).
33. Tian et al., Cell metabolism, 23:165-78 (2015).
34. Saely et al., Gerontology 58, 15-23 (2012)10.1159/000321319).

In view of the many possible embodiments to which the principles of the disclosure may be applied, it should be recognized that the illustrated embodiments are only examples of the disclosure and should not be taken as limiting the scope of the invention. Rather, the scope of the invention is defined by the following claims. We therefore claim as our invention all that comes within the scope and spirit of these claims.

We claim:

1. A method of increasing thermogenesis in a subject, comprising:
administering a therapeutically effective amount of one or more agents that increase ERRγ activity, thereby increasing thermogenesis in the subject.

2. The method of claim 1, wherein the subject is obese or has reduced thermogenesis due to increased age.

3. The method of claim 1, wherein the subject is at least 65 years old, at least 70 years old, or at least 75 years old.

4. The method of claim 1, wherein the subject is a human subject or a veterinary subject.

5. The method of claim 1, wherein the subject is obese, and the method reduces the body mass index (BMI) of the subject.

6. The method of claim 1, wherein the subject is or is at risk for hypothermia.

7. The method of claim 1, wherein the method increases fatty acid uptake and/or oxidation in brown adipose tissue (BAT).

8. The method of any of claim 1, wherein the one or more agents that increase ERRγ activity comprises:
a nucleic acid molecule encoding ERRγ;
one or more ERRγ agonists;
an ERRγ protein; or
combinations thereof.

9. The method of claim 1, wherein the one or more agents that increase ERRγ activity is: 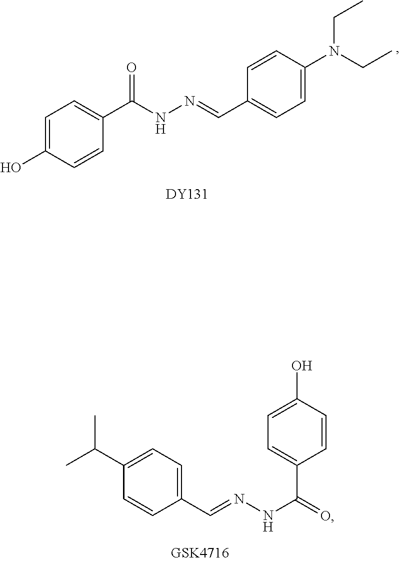
or combinations thereof.

10. The method of claim 1, wherein the one or more agents that increase ERRγ activity is:

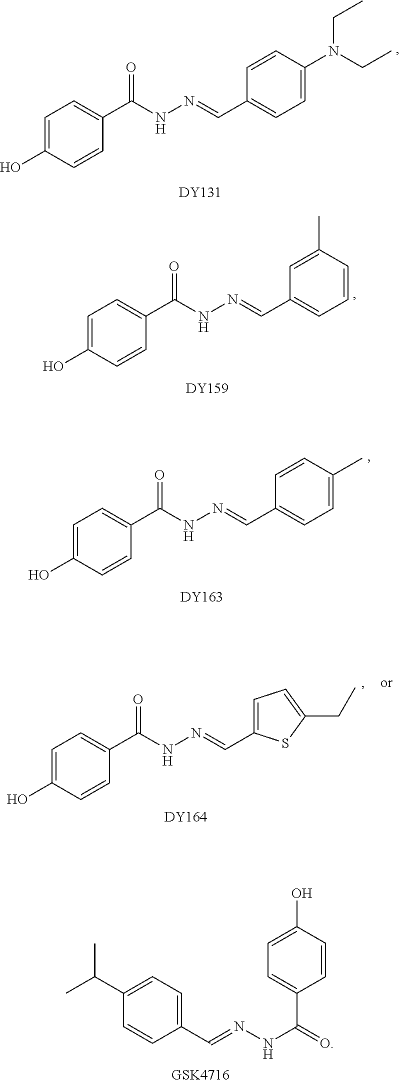

11. The method of claim 1, wherein the one or more agents that increase ERRγ activity is: 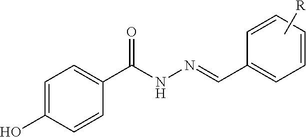
wherein R is H (DY162), p-CH₃ (DY163), 2-Cl, 3-CF₃ (DY165), p-CF₃ (DY168), p-OCH₃ (DY169), 3-NO₂, 4CF₃ (DY170), 2,3-O₂CH₃(DY174), or m-CH₃ (DY159), 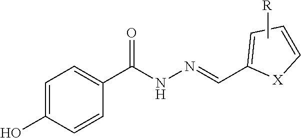
wherein X is S and R is 5-CH₃ (DY166), 5-CH₂CH₃ (DY164), or 5-NO₂ (DY167); wherein X is O and R is 4,5-CH₃ (DY173) or CH₂CH₃ (DY175), or wherein X is CH, and R is 2-C1, 3-CF₃, p-CF₃; p-OCH₃, 3-NO₂, 4-CF₃; or 2,3-O₂CH₃; 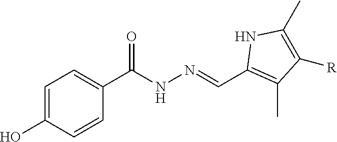
wherein R is H (DY117) or R is Br (DY172), 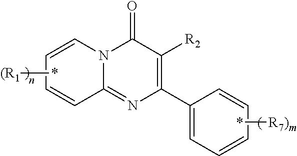
wherein
m is 0, 1 or 2;
n is 0, 1 or 2;
R₁ and R₇ are independently selected from
H;
2) Halo;
3) OH;
4) (C=0)ₐ, O_{b}C₁-C₄ alkyl, wherein a is 0 or 1 and b is 0 or 1, wherein the alkyl can be substituted by 0, 1 or more substituted groups independently selected from H or C₃C₆ heterocyclyl;
5) (C=0), O_{b}C₃-C₆ cycloalkyl, wherein a is 0 or 1 and b is 0 or 1;
R2 is selected from:
1) H;
2) C₁-C₃ alkyl, wherein the alkyl can be substituted by 0, 1 or more substituted groups independently selected from H or C₃C₆ heterocyclyl;
3) C₃-C₆ cycloalkyl; 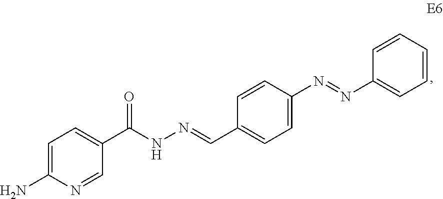 [SMILES: Nc1ccc(C(=O)N/N=C/c2ccc(/N=N/c3ccccc3)cc2)cn1]
or combinations thereof.

12. The method of claim 8, wherein the nucleic acid molecule encoding ERRγ comprises a sequence having at least 90% sequence identity to SEQ ID NO: 1.

13. The method of claim 8, wherein the nucleic acid molecule encoding ERRγ comprises a vector.

14. The method of claim 13, wherein the vector comprises a viral vector.

15. The method of claim 8, wherein the nucleic acid molecule encoding ERRγ is operably linked to a promoter.

16. The method of claim 8, wherein the ERRγ protein comprises a sequence having at least 90% sequence identity to SEQ ID NO: 2.

17. The method of claim 1, further comprising administering a therapeutically effective amount of one or more beta adrenergic agonists to the subject.

18. The method of claim 17, wherein the one or more beta adrenergic agonists comprise a beta-2 agonist.

19. The method of claim 18, wherein the beta-2 agonist is epinephrine, norepinephrine, isoproterenol, GSK-159797, GSK-597901, GSK-159802, GSK-642444, GSK-678007, or combinations thereof.

20. The method of claim 18, wherein the one or more beta adrenergic agonists comprise a beta-3 agonist.

21. The method of claim 20, wherein the beta-3 agonist is amibegron (SR-58611A), CL-316,243, L-742,791, L-796,568, LY-368,842, mirabegron (YM-178), Ro40-2148, solabegron (GW-427,353), BRL 37344, ICI 215,001, L-755,507, ZD 2079, ZD 7114, or combinations thereof.

22. A composition comprising:
one or more agents that increase ERRγ activity; and
one or more beta adrenergic agonists.
